(19) United States
(12) Patent Application Publication
(10) Pub. No.: US 2006/0229324 A1
(43) Pub. Date: Oct. 12, 2006

(54) Quinazolin-4-one derivatives

(76) Inventors: Akiko Itai, Tokyo (JP); Susumu Muto, Tokyo (JP); Tsuyoshi Inoue, Osaka (JP); Yoshihiro Urade, Kyoto (JP)

Correspondence Address: GREENBLUM & BERNSTEIN, P.L.C., 1950 ROLAND CLARKE PLACE, RESTON, VA, 20191, US

(21) Appl. No.: 10/529,946

(22) PCT Filed: Oct. 2, 2003
(86) PCT No.: PCT/JP03/12648

§ 371 Date: Oct. 4, 2005

(30) Foreign Application Priority Data
Oct. 3, 2002 (JP) 2002-291114

(51) Int. Cl. A61K 31/517; C07D 403/02

(52) U.S. Cl. 514/266.23; 544/284

(57) ABSTRACT

A medicament having an inhibitory activity against hematopoietic prostaglandin D2 synthase, which comprises as an active ingredient a compound represented by the following general formula (I) or a salt thereof: 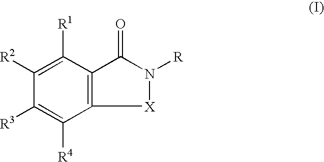
wherein X represents a group represented by the formula —N═C(R⁵)— or the formula —NH—CH(R⁵)—, R¹, R², R³, and R⁴ represent a hydrogen atom, a halogen atom, a C₁ to C₆ alkyl group, or a hydroxy group, R⁵ represents a C₁ to C₆ alkyl group or a C₆ to C₁₀ aryl group, and R represents an amino group.

### FIELD OF INVENTION

The present invention relates to medicaments having inhibitory activity against hematopoietic prostaglandin D2 synthase, and also having actions such as antiallergic action, anti-inflammatory action, protection against tissue damage, or antiasthmatic action.

### BACKGROUND ART

Prostaglandin D2 (PGD2) is a kind of arachidonic acid metabolite, which is synthesized via the cyclooxygenase pathway in the arachidonate cascade with the prostaglandin H2 (PGH2) being an intermediate. From this sort of biosynthetic pathway, it is known that prostaglandin F2 α (PGF2 α), prostaglandin E2 (PGE2), prostaglandin I2 (PGI2), and thromboxane A2 (TXA2) are synthesized besides PGD2. As for allergic inflammatory diseases such as asthma and allergic rhinitis, in a mast cell to which a complex of an antigen and immunoglobulin E (IgE), which is suggested to play a primary role in an allergic reaction, is bound for activation, it is considered that the arachidonic acid metabolic cascade is activated and various kinds of inflammatory mediators derived from arachidonic acid are released which have important roles for inductions of allergic symptoms.

Among them, PGD2 is an inflammatory mediator which is produced and released most abundantly, and is detected in a bronchoalveolar lavage fluid of asthmatic patients at a high concentration (The Journal of Immunology, (USA), 1982, Vol. 129, No. 4, p. 1627-1631; The New England Journal of Medicine, (USA), 1986, Vol. 315, No. 13, p. 800-804). Since PGD2 not only exhibits a strong contracting effect on airway, but also has an activating effect of eosinophil involved deeply in inflammation and an inducing effect of airway hypersensitiveness, PGD2 is considered to be closely related in pathologic conditions of allergic asthma among allergic inflammatory diseases (The Journal of Immunology, (USA), 1982, Vol. 129, No. 4, p. 1627-1631; The New England Journal of Medicine, (USA), 1986, Vol. 315, No. 13, p. 800-804; The New England Journal of Medicine, (USA), 1984, Vol. 311, No. 4, p. 209-213; The Journal of Immunology, (USA), 1992, Vol. 148, No. 11, p. 3536-3542; Science, (USA), 2000, Vol. 287, p. 2013-2017).

As synthases that synthesize PGD2 from PGH2, two kinds enzymes that are hematopoietic-type (H-PGDS) and lipocalin-type are known. Since the lipocalin-type enzyme distributes mainly in the brain and PGD2 is a sleep-inducing substance, it is known that the enzyme is known to be involved in induction of sleep, reduction of body temperature, suppression of progestin secretion, and response regulatory action of pain and smell (Vitamins and hormones, (USA), 2000, Vol. 58, p. 89-120; The Journal of Biological Chemistry, (USA), 1985, Vol. 260, No. 23, p. 12140-12145; Biochemica et Biophysica Acta, (Netherlands), 2000, Vol. 1482, No. 1-2, p. 259-271), and in particular, the relation with sleep regulatory action has been focused. Whilst, since the hematopoietic-type enzyme distributes mainly in placenta, lungs, mast cells, and antigen presenting cells, the enzyme is considered to be mainly involved in allergic inflammatory diseases (The Journal of Immunology, (USA), 1989, Vol. 143, No. 9, p. 2982-2989; The Journal of Biological Chemistry, (USA), 1990, Vol. 265, No. 1, p. 371-375; The Journal of Biological Chemistry, (USA), 1995, Vol. 270, No. 7, p. 3239-3246).

As an inhibitor to the hematopoietic-type enzyme, HQL-79 (4-benzhydryloxy-1-{3-(1H-tetrazol-5-yl)-propyl}pyridine) that is a benzhydryloxy derivative having a tetrazole skeleton is known. It is reported that HQL-79 inhibits inflammatory pathologic conditions of airway such as eosinophilic infiltration into airway and delayed asthmatic response in an asthma pathological model (Japanese Journal of Pharmacology, 1998, Vol. 78, No. 1, p. 1-10; Japanese Journal of Pharmacology, 1998, Vol. 78, No. 1, p. 11-22). However, its activity is not satisfactory.

At present, antiallergic agents such as ketotifen and terfenazine, antihistamic agents such as chlorpheniramine maleate, and anti-inflammatory steroids are used for allergic diseases. However, conventional antiallergic agents and antihistamic agents sometimes fail to have sufficient pharmacological effects, and fail to sufficiently suppress delayed allergic reaction. They also have problems of central side effects such as drowsiness and sedative symptom. For the inhibition of the delayed allergic reactions, anti-inflammatory steroids are effective. However, they have problems of side effects such as immune suppression. Accordingly, they are not medicaments that can be used easily and safely. Therefore, selective and strong inhibitors against the hematopoietic-type enzymes are expected to be potent therapeutic agents for allergic inflammatory diseases, particularly allergic asthma, with more reduced side effects compared with conventional drugs.

### DISCLOSURE OF THE INVENTION

An object of the present invention is to develop selective inhibitors against hematopoietic-type PGD2 synthase and to provide superior medicaments with reduced side effects and high safety, for the treatment of allergic inflammatory diseases such as allergic rhinitis, particularly for the treatment of allergic asthma.

Hematopoietic-type PGD2 synthase is a glutathione-demanding enzyme which is classified as σ class GST, a subtype of Glutathione S-transferase (GST). Recently, its three-dimensional structure was elucidated by X-ray structure analysis (Cell, (USA), 1997, Vol. 90, No. 6, p. 1085-1095), and it was reported that this enzyme has a shorter fourth α helix and a specific wide and deep Cleft structure compared with other GSTs. Based on the elucidated three-dimensional structure, the inventors of the present invention designed and synthesized organic compounds having a low molecular weight that are expected to bindable to a moiety of the Cleft structure of the hematopoietic-type PGD2 synthase. Further, according to a strategy led by the molecular design, the inventors carried out derivatization of classes of compounds which were found to have desired enzyme inhibitory activities. As a result, they found that the compounds represented by the following general formula (I) have extremely superior inhibitory activity against the hematopoietic-type PGD2 synthase (H-PGDS). The present invention was achieved on the basis of these findings.

The present invention thus provides a medicament having inhibitory activity against hematopoietic PGD2 synthase, which comprises as an active ingredient a substance selected from the group consisting of a compound represented by the following general formula (I) and a pharmacologically acceptable salt thereof, and a hydrate thereof and a solvate thereof: 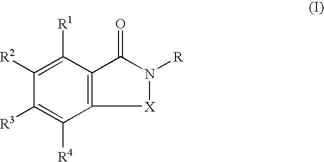
wherein X represents a group represented by the formula —N═C(R⁵)— (wherein a bond at the left end binds to the benzene ring and a bond at the right end binds to the nitrogen atom), or the formula —NH—CH(R⁵)— (wherein a bond at the left end binds to the benzene ring and a bond at the right end binds to the nitrogen atom), R¹, R², R³, and R⁴ independently represent a hydrogen atom, a halogen atom, a C₁ to C₆ alkyl group which may be substituted, or a hydroxy group which may be substituted, R⁵ represents a C₁ to C₆ alkyl group which may be substituted, or a C₆ to C₁₀ aryl group which may be substituted,
R represents an amino group which may be substituted.

According to preferred embodiments of the aforementioned invention, provided are the aforementioned medicament wherein R is a group represented by the following general formula (II): 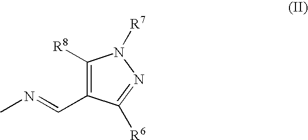
wherein R⁶ represents a C₁ to C₁₀ alkyl group which may be substituted, or a C₆ to C₁₀ aryl group which may be substituted,
R⁷ represents a C₁ to C₆ alkyl group which may be substituted, or a C₆ to C₁₀ aryl group which may be substituted,
R⁸ represents a halogen atom, hydroxy group, or a C₁ to C₆ alkoxy group which may be substituted;
the aforementioned medicament wherein X is a group represented by the formula —N═C(R⁵)— (wherein a bond at the left end binds to the benzene ring and a bond at the right end binds to the nitrogen atom);
the aforementioned medicament wherein R¹, R², R³, and R⁴ independently represent a hydrogen atom, a halogen atom, a C₁ to C₆ alkyl group, or a C₁ to C₆ alkoxy group; the aforementioned medicament wherein R⁵ is a C₁ to C₆ alkyl group which may be substituted with a group selected from the following substituent group α-1, or a phenyl group which may be substituted with a group selected from the following substituent group α-1;
[Substituent Group α-1] hydroxy group, C₁ to C₆ alkoxy group
the aforementioned medicament wherein R⁶ is a C₁ to C₁₀alkyl group which may be substituted with a group selected from the following substituent group α-2, or a phenyl group which may be substituted with a C₁ to C₆ alkyl group;
[Substituent Group α-2] halogen atoms, carboxy group, carbamoyl group, C₁ to C₆ alkoxycarbonyl group
the aforementioned medicament wherein R⁷ is a C₁ to C₆ alkyl group, or a phenyl group which may be substituted with a group selected from the following substituent group α-3;
[Substituent Group α-3] halogen atoms, C₁ to C₆ alkyl group, C₁ to C₆ alkoxy group, nitro group
and the aforementioned medicament wherein R⁸ is a halogen atom, hydroxy group, or a C₁ to C₆ alkoxyl group which may be substituted with a group selected from the following substituent group α-4.
[Substituent Group α-4] carboxy group, C₁ to C₆ alkoxycarbonyl group

Furthermore, according to preferred embodiments of the aforementioned invention, provided are the aforementioned medicament having one or more actions selected from the group consisting of antiallergic action, antiallergic inflammation, and antiasthmatic action; the aforementioned medicament having an action of preventing the aggravation of brain damage, and/or an action of improving the prognosis of brain damage; the aforementioned medicament having an action of cerebroprotection; and the aforementioned medicament having one or more actions selected from the group consisting of an action of regulating estrous cycle, an action of regulating sleep, an action of thermoregulation, an analgesic action, and an action of regulating olfaction.

Form another aspect, the present invention provides use of the substance selected from the group consisting of the compound represented by the aforementioned general formula (I) and the pharmacologically acceptable salt thereof, and the hydrate thereof and the solvate thereof for manufacture of the aforementioned medicament.

From further another aspect, the present invention provides a method for inhibiting hematopoietic PGD2 synthase in mammal including a human, which comprises the step of administering an effective amount of the substance selected from the group consisting of the compound represented by the aforementioned general formula (I) and the pharmacologically acceptable salt thereof, and the hydrate thereof and the solvate thereof to a mammal including a human; a method for preventive and/or therapeutic treatment of one or more diseases selected from the group consisting of allergic disease, allergic inflammatory disease, and asthma, which comprises the step of administering a preventively and/or therapeutically effective amount of the substance selected from the group consisting of the compound represented by the aforementioned general formula (I) and the pharmacologically acceptable salt thereof, and the hydrate thereof and the solvate thereof to a mammal including a human; a method for preventing the aggravation of brain damage, which comprises the step of administering an effective amount of the substance selected from the group consisting of the compound represented by the aforementioned general formula (I) and the pharmacologically acceptable salt thereof, and the hydrate thereof and the solvate thereof to a mammal including a human; a method for improving the prognosis of brain damage, which comprises the step of administering an effective amount of the substance selected from the group consisting of the compound represented by the aforementioned general formula (I) and the pharmacologically acceptable salt thereof, and the hydrate thereof and the solvate thereof to a mammal including a human; a method for cerebroprotection, which comprises the step of administering an effective amount of the substance selected from the group consisting of the compound represented by the aforementioned general formula (I) and the pharmacologically acceptable salt thereof, and the hydrate thereof and the solvate thereof to a mammal including a human; a method for regulating biological actions selected from the group consisting of estrous cycle, sleep, body temperature, pain sensation, and olfaction, which comprises the step of administering an effective amount of the substance selected from the group consisting of the compound represented by the aforementioned general formula (I) and the pharmacologically acceptable salt thereof, and the hydrate thereof and the solvate thereof to a mammal including a human.

Furthermore, the present invention provides a compound represented by the general formula (I-1) or a salt thereof, or a hydrate thereof or a solvate thereof: 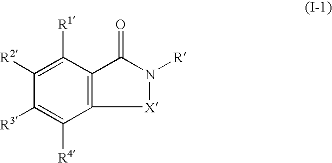
wherein X′ represents a group represented by the formula —N═C(R⁵′)— (wherein a bond at the left end binds to the benzene ring and a bond at the right end binds to the nitrogen atom), or the formula —NH—CH(R⁵′)— (wherein a bond at the left end binds to the benzene ring and a bond at the right end binds to the nitrogen atom), R¹′, R²′, R³′, and R⁴′ independently represent a hydrogen atom, a halogen atom, a C₁ to C₆ alkyl group which may be substituted, or a hydroxy group which may be substituted,
R⁵′ represents a C₁ to C₆ alkyl group which may be substituted, or a C₆ to C₁₀ aryl group which may be substituted,
R′ represents an amino group which may be substituted,
provided that the compounds represented by the following compound group β are excluded.

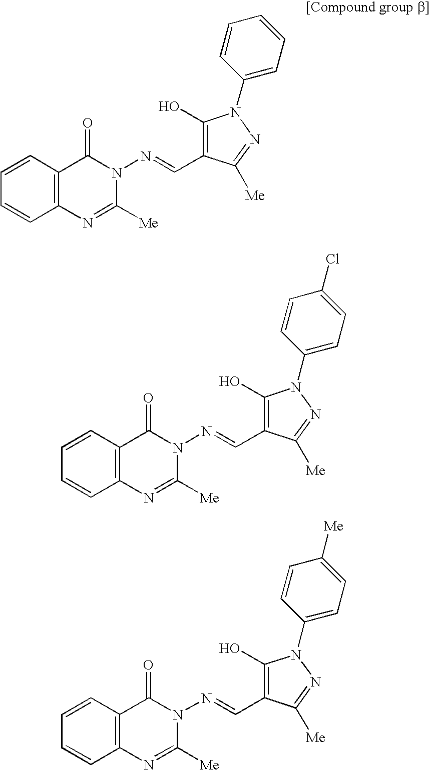

Furthermore, according to preferred embodiments of the aforementioned invention, provided are the aforementioned compound or a salt thereof, or a hydrate thereof or a solvate thereof, wherein R′ is represented by the following general formula (II-1): 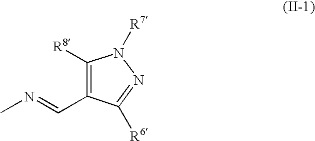
wherein R⁶′ represents a C₁ to C₁₀alkyl group which may be substituted, or a phenyl group which may be substituted with a C₁ to C₆ alkyl group,
R⁷′ represents a C₁ to C₆ alkyl group which may be substituted, or a C₆ to C₁₀ aryl group which may be substituted,
R⁸′ represents a halogen atom, hydroxy group, or a C₁ to C₆ alkoxy group which may be substituted.

### BEST MODE FOR CARRYING OUT THE INVENTION

The terms used in the present specification have the following meanings.

As the halogen atom, any of fluorine atom, chlorine atom, bromine atom, or iodine atom may be used unless otherwise specifically referred to.

The “C₁ to C₁₀alkyl group” may be straight chain, branched chain, cyclic, and combination of these unless otherwise specifically referred to. More specifically, examples include methyl group, ethyl group, n-propyl group, isopropyl group, cyclopropyl group, n-butyl group, isobutyl group, sec-butyl group, tert-butyl group, cyclobutyl group, cyclopropylmethyl group, n-pentyl group, isopentyl group, neopentyl group, tert-pentyl group, cyclopentyl group, n-hexyl group, cyclohexyl group, 3,3-dimethylbutyl group, 2-ethylbutyl group, 2-methylpentyl group, 3-methylpentyl group, 4-methylpentyl group, or 1-adamantyl group. An alkyl moiety of other substituents containing the alkyl moiety has the same meaning.

The “C₆ to C₁₀ aryl group” may either be monocyclic or fused cyclic. Examples include phenyl group, 1-naphthyl group, and 2-naphthyl group.

Examples of the “C₁ to C₆ alkoxy group” include methoxy group, ethoxy group, n-propoxy group, or tert-butoxy group.

Examples of the “C₁ to C₆ alkoxycarbonyl group” include methoxycarbonyl group, ethoxycarbonyl group, n-propoxycarbonyl group, or tert-butoxycarbonyl group.

Examples of the “C₁ to C₆ alkylideneamino group” include methylideneamino group, ethylideneamino group, n-propylideneamino group, or tert-butylideneamino group.

In the present specification, when a certain functional group is defined as “which may be substituted,” the definition means that the functional group may sometimes have one or more substituents at chemically substitutable positions. Kind of substituents, number of substituents, and the position of substituents existing in the functional groups are not particularly limited, and when two or more substituents exist, they may be the same or different. Examples of the substituent existing in the functional group include, for example, halogen atoms, oxo group, thioxo group, nitro group, nitroso group, cyano group, isocyano group, cyanato group, thiocyanato group, isocyanato group, isothiocyanato group, hydroxy group, sulfanyl group, carboxy group, sulfanylcarbonyl group, oxalo group, methooxalo group, thiocarboxy group, dithiocarboxy group, carbamoyl group, thiocarbamoyl group, sulfo group, sulfamoyl group, sulfino group, sulfinamoyl group, sulfeno group, sulfenamoyl group, phosphono group, hydroxyphosphonyl group, a C₁ to C₆ alkyl group, a C₂ to C₆ alkenyl group whose examples include, for example, vinyl group, allyl group, and 1-propenyl group, a C₂ to C₆ alkynyl group whose examples include, for example, ethynyl group and 1-propynyl group, a C₁ to C₆ alkylidene group, a C₆ to C₁₀ aryl group, a C₇ to C₁₂ aralkyl group whose examples include, for example, benzyl group, phenethyl group, 1-naphthylmethyl group, and 2-naphthylmethyl group, a C₇ to C₁₂ aralkylidene group whose examples include, for example, benzylidene group, phenethylidene group, 1-naphthylmethylidene group, and 2-naphthylmethylidene group, a C₁ to C₆ alkoxy group, a C₆ to C₁₀ aryloxy group whose examples include, for example, phenoxy group, 1-naphthyloxy group, and 2-naphthyloxy group, a C₇ to C₁₂ aralkyloxy group whose examples include, for example, benzyloxy group, (1-naphthylmethyl)oxy group, and (2-naphthylmethyl)oxy group, a C₁ to C₆ alkylsulfanyl group whose examples include, for example, methylsulfanyl group and ethylsulfanyl group, a C₆ to C₁₀ arylsulfanyl group whose examples include, for example, phenylsulfanyl group, 1-naphthylsulfanyl group, and 2-naphthylsulfanyl group, a C₇ to C₁₂ aralkyloxysulfanyl group whose examples include, for example, benzylsulfanyl group, (1-naphthylmethyl)sulfanyl group, and (2-naphthylmethyl)sulfanyl group, a C₁ to C₆ alkanoyl group whose examples include, for example, acetyl group, propionyl group, n-butyryl group, and pivaloyl group, a C₆ to C₁₀ aroyl group whose examples include, for example, benzoyl group, 1-naphthoyl group, and 2-naphthoyl group, a C₁ to C₆ alkylsulfonyl group whose examples include, for example, methanesulfonyl group, ethanesulfonyl group, and propanesulfonyl group, a C₆ to C₁₀ arylsulfonyl group whose examples include, for example, benzenesulfonyl group, 1-naphthalenesulfonyl group, and 2-naphthalenesulfonyl group, a C₁ to C₆ alkoxycarbonyl group, amino group, hydrazino group, hydrazono group, diazenyl group, ureido group, thioureido group, guanidino group, carbamoimidoyl group (amidino group), azido group, imino group, hydroxyamino group, hydroxyimino group, aminooxy group, diazo group, semicarbazino group, semicarbazono group, allophanyl group, hydantoyl group, phosphano group, phosphoroso group, phospho group, boryl group, silyl group, stannyl group, selanyl group, oxido group, or the 4 to 10-membered monocyclic, bicyclic or more polycyclic and unsaturated, partly saturated, or completely saturated heterocyclic group, which comprises 1 to 4 hetero atoms selected from the group consisting of nitrogen atom, oxygen atom, and sulfur atom, whose examples include, for example, thienyl group, furyl group, pyrrolyl group, oxazolyl group, isoxazolyl group, thiazolyl group, isothiazolyl group, imidazolyl group, pyrazolyl group, benzothiophenyl group, benzofuranyl group, isobenzothiophenyl group, isobenzofuranyl group, indolyl group, isoindolyl group, indolizinyl group, 1H-indazolyl group, purinyl group, benzothiazolyl group, benzoxazolyl group, benzimidazolyl group, 1,2,3-thiadiazolyl group, 1,2,4-thiadiazolyl group, 1,3,4-thiadiazolyl group, 1,3,4-oxadiazolyl group, 1,2,3-triazolyl group, 1,2,4-triazolyl group, tetrazolyl group, chromenyl group, pyridyl group, pyridazinyl group, pyrimidinyl group, pyrazinyl group, quinolizinyl group, quinolyl group, isoquinolyl group, phthalazinyl group, naphthyridinyl group, quinoxalinyl group, quinazolinyl group, cinnolinyl group, pteridinyl group, 1,2,4-triazinyl group, chromanyl group, isochromanyl group, azetidinyl group, 2-oxoazetidinyl group, pyrrolidinyl group, pyrrolinyl group, imidazolidinyl group, imidazolinyl group, pyrazolidinyl group, pyrazolinyl group, piperidyl group, piperazinyl group, morpholino group, morpholinyl group, thiomorpholino group, thiomorpholinyl group, indolinyl group, isoindolinyl group, 1,2,3,4-tetrahydroquinolyl group, and quinuclidinyl group.

These substituents may further be substituted with one or more kinds of other substituents. Examples include a C₁ to C₆ halogenated alkyl group whose examples include, for example, chloromethyl group, dichloromethyl group, trichloromethyl group, difluoromethyl group, trifluoromethyl group, 2,2,2-trifluoroethyl group, and pentafluoroethyl group, a C₁ to C₆ halogenated alkoxy group whose examples include, for example, trifluoromethoxy group and pentafluoroethoxy group, a carboxy-substituted C₁ to C₆ alkyl group whose examples include, for example, carboxymethyl group and carboxyethyl group, a C₁ to C₆ alkyl-substituted amino group whose examples include, for example, methylamino group and ethylamino group, a heterocyclic ring-substituted C₁ to C₆ alkylidene group (said heterocyclic ring represents the aforementioned “4 to 10-membered monocyclic, bicyclic or more polycyclic and unsaturated, partly saturated, or completely saturated heterocyclic group, which comprises 1 to 4 hetero atoms selected from the group consisting of nitrogen atom, oxygen atom, and sulfur atom.” A heterocyclic ring explained as follows has the same meaning unless otherwise specifically referred to. Examples include, for example, (thiophen-2-yl)methylidene group, (pyridin-3-yl)methylidene group, and (pyrazol-4-yl)methylidene group), and a heterocyclic ring-carbonyl group whose examples include, for example, (thiophen-2-yl)carbonyl group, nicotinoyl group, and (pyrazol-4-yl)carbonyl group. Furthermore, two or more substituents of the aforementioned substituents may form a ring together with the atoms to which they bind (carbon atom, nitrogen atom, boron atom, and the like). In these rings, one or more hetero atoms selected from the group consisting of nitrogen atom, oxygen atom, and sulfur atom may be included as ring-constituting atoms, and one or more substituents may exist on the ring. The ring may either be monocyclic or fused cyclic, or may be unsaturated, partly saturated, or completely saturated.

In the aforementioned general formula (I), examples of X include the formula —N═C(R⁵)— (wherein a bond at the left end binds to the benzene ring and a bond at the right end binds to the nitrogen atom), and the formula —NH—CH(R⁵)— (wherein a bond at the left end binds to the benzene ring and a bond at the right end binds to the nitrogen atom). The formula —N═C(R⁵)— (wherein a bond at the left end binds to the benzene ring and a bond at the right end binds to the nitrogen atom) is preferred.

Examples of the substituent, according to “a C₁ to C₆ alkyl group which may be substituted” and “a hydroxy group which may be substituted” in the definition of R¹, R², R³, and R⁴ include similar groups to the substituents in the definition of the aforementioned “which may be substituted.”

Examples of R¹, R², R³, and R⁴ independently include hydrogen atom, halogen atom, a C₁ to C₆ alkyl group which may be substituted, and a hydroxy group which may be substituted. Hydrogen atom, halogen atom, methyl group, and methoxy group are preferred,

(1) R¹, R², R³, and R⁴ are all hydrogen atoms,

(2) R¹ is methyl group, and R², R³, and R⁴ are hydrogen atoms,

(3) R¹, R², and R³ are hydrogen atoms, and R⁴ is methyl group,

(4) R¹, R³, and R⁴ are hydrogen atoms, and R² is chloro group,

(5) R¹, R³, and R⁴ are hydrogen atoms, and R² is bromo group,

(6) R¹, R², and R⁴ are hydrogen atoms, and R³ is chloro group, and

(7) R¹ and R⁴ are hydrogen atoms, and R² and R³ are methoxy group,

are more preferred, and,

(1) R¹, R², R³, and R⁴ are all hydrogen atoms, and

(2) R¹, R², and R³ are hydrogen atoms, and R⁴ is methyl group,

are further more preferred.

Examples of the substituent, according to “a C₁ to C₆ alkyl group which may be substituted” and “a C₆ to C₁₀ aryl group which may be substituted” in the definition of R⁵ include similar groups to the substituents in the definition of the aforementioned “which may be substituted.”

Examples of R⁵ include a C₁ to C₆ alkyl group which may be substituted, and a C₆ to C₁₀ aryl group which may be substituted. Methyl group, ethyl group, isopropyl group, n-butyl group, tert-butyl group, hydroxymethyl group, methoxymethyl group, phenyl group, 2-methoxyphenyl group, 3-methoxyphenyl group, and 4-methoxyphenyl group are preferred, and methyl group, hydroxymethyl group, and 4-methoxyphenyl group are more preferred. When X is the formula —NH—CH(R⁵)— (wherein a bond at the left end binds to the benzene ring and a bond at the right end binds to the nitrogen atom), it is particularly preferable that R⁵ is hydroxymethyl group.

Examples of the substituent, according to “an amino group which may be substituted” in the definition of R include similar groups to the substituents in the expression of “which may be substituted.”

Examples of R include an amino group which may be substituted. A C₁ to C₆ alkylideneamino group is preferred. The group represented by the following general formula (II): 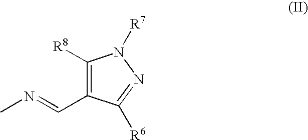
wherein R⁶ represents a C₁ to C₁₀alkyl group which may be substituted, or a C₆ to C₁₀ aryl group which may be substituted,
R⁷ represents a C₁ to C₆ alkyl group which may be substituted, or a C₆ to C₁₀ aryl group which may be substituted,
R⁸ represents a halogen atom, hydroxy group, or a C₁ to C₆ alkoxy group which may be substituted, and the groups represented by the following formulas: 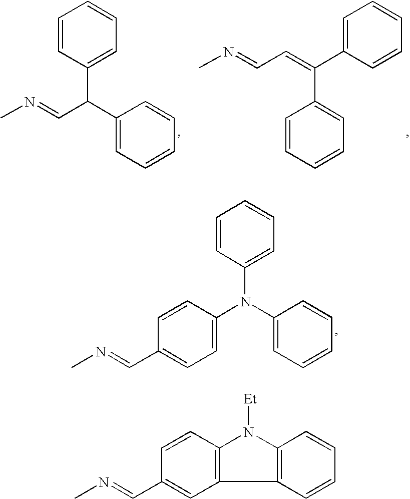
are more preferred. The group represented by the following general formula (II): 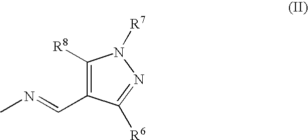
wherein R⁶ represents a C₁ to C₁₀alkyl group which may be substituted, or a C₆ to C₁₀ aryl group which may be substituted,
R⁷ represents a C₁ to C₆ alkyl group which may be substituted, or a C₆ to C₁₀ aryl group which may be substituted,
R⁸ represents a halogen atom, hydroxy group, or a C₁ to C₆ alkoxy group which may be substituted, is further more preferred.

Examples of the substituent, according to “a C₁ to C₁₀alkyl group which may be substituted” and “a C₆ to C₁₀ aryl group which may be substituted” in the definition of R⁶ include similar groups to the substituents in the expression of “which may be substituted.”

Examples of R⁶ include a C₁ to C₁₀alkyl group which may be substituted and a C₆ to C₁₀ aryl group which may be substituted. Methyl group, ethyl group, trifluoromethyl group, 2-(ethoxycarbonyl)ethyl group, 2-carboxyethyl group, 2-carbamoylethyl group, 3-(ethoxycarbonyl)propyl group, 3-carboxypropyl group, 3-carbamoylpropyl group, 1-adamantyl group, and 4-tert-butylphenyl group are preferred, and methyl group, trifluoromethyl group, 3-(ethoxycarbonyl)propyl group, and 3-carboxypropyl group are more preferred.

Examples of the substituent, according to “a C₁ to C₆ alkyl group which may be substituted” and “a C₆ to C₁₀ aryl group which may be substituted” in the definition of R⁷ include similar groups to the substituents in the expression of “which may be substituted.”

Examples of R⁷ include a C₁ to C₆ alkyl group which may be substituted and a C₆ to C₁₀ aryl group which may be substituted. Methyl group, phenyl group, 2-chlorophenyl group, 3-chlorophenyl group, 4 chlorophenyl group, 4-methylphenyl group, 4-methoxyphenyl group, and 4-nitrophenyl group are preferred, and phenyl group, 3-chlorophenyl group, and 4-nitrophenyl group are more preferred.

Examples of the substituent, according to “a C₁ to C₆ alkoxy group which may be substituted” in the definition of R⁸ include similar groups to the substituents in the expression of “which may be substituted.”

Examples of R⁸ include halogen atom, hydroxy group, and a C₁ to C₆ alkoxy group which may be substituted. Halogen atom, hydroxy group, (ethoxycarbonyl)methoxy group, and carboxymethoxy group are preferred, and hydroxy group and carboxymethoxy group are more preferred.

In the aforementioned general formula (I), examples of X′ include the formula —N═C(R⁵′)— (wherein a bond at the left end binds to the benzene ring and a bond at the right end binds to the nitrogen atom), and the formula —NH—CH(R⁵′)— (wherein a bond at the left end binds to the benzene ring and a bond at the right end binds to the nitrogen atom). The formula —N═C(R⁵′)— (wherein a bond at the left end binds to the benzene ring and a bond at the right end binds to the nitrogen atom) is preferred.

Examples of the substituent, according to “a C₁ to C₆ alkyl group which may be substituted” and “a hydroxy group which may be substituted” in the definition of R¹′, R²′, R³′, and R⁴′ include similar groups to the substituents in the expression of the aforementioned “which may be substituted.”

Examples of R¹′, R²′, R³′, and R⁴′ independently include hydrogen atom, halogen atom, a C₁ to C₆ alkyl group which may be substituted, and a hydroxy group which may be substituted. Hydrogen atom, halogen atom, methyl group, and methoxy group are preferred,

(1) R¹′, R²′, R³′, and R⁴′ are all hydrogen atoms,

(2) R¹′ is methyl group, and R²′, R³′, and R⁴′ are hydrogen atoms,

(3) R¹′, R²′, and R³′ are hydrogen atoms, and R⁴′ is methyl group,

(4) R¹′, R³′, and R⁴′ are hydrogen atoms, and R²′ is chloro group,

(5) R¹′, R³′, and R⁴′ are hydrogen atoms, and R²′ is bromo group,

(6) R¹′, R²′, and R⁴′ are hydrogen atoms, and R³′ is chloro group, and

(7) R¹′ and R⁴′ are hydrogen atoms, and R²′ and R³′ are methoxy group,

are more preferred, and,

(1) R¹′, R²′, R³′, and R⁴′ are all hydrogen atoms, and

(2) R¹′, R²′, and R³′ are hydrogen atoms, and R⁴′ is methyl group,

are further more preferred.

Examples of the substituent, according to “a C₁ to C₆ alkyl group which may be substituted” and “a C₆ to C₁₀ aryl group which may be substituted” in the definition of R⁵′ include similar groups to the substituents in the expression of the aforementioned “which may be substituted.”

Examples of R⁵′ include a C₁ to C₆ alkyl group which may be substituted, and a C₆ to C₁₀ aryl group which may be substituted. Methyl group, ethyl group, isopropyl group, n-butyl group, tert-butyl group, hydroxymethyl group, methoxymethyl group, phenyl group, 2-methoxyphenyl group, 3-methoxyphenyl group, and 4-methoxyphenyl group are preferred, and methyl group, hydroxymethyl group, and 4-methoxyphenyl group are more preferred. When X′ is the formula —NH—CH(R⁵′)— (wherein a bond at the left end binds to the benzene ring and a bond at the right end binds to the nitrogen atom), it is particularly preferable that R⁵′ is hydroxymethyl group.

Examples of the substituent, according to “an amino group which may be substituted” in the expression of R′ include similar groups to the substituents in the expression of “which may be substituted.”

Examples of R′ include an amino group which may be substituted. A C₁ to C₆ alkylideneamino group is preferred. The group represented by the following general formula (II-1): 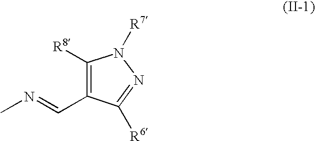
wherein R⁶′ represents a C₁ to C₁₀alkyl group which may be substituted, or a C₆ to C₁₀ aryl group which may be substituted,
R⁷′ represents a C₁ to C₆ alkyl group which may be substituted, or a C₆ to C₁₀ aryl group which may be substituted,
R⁸′ represents a halogen atom, hydroxy group, or a C₁ to C₆ alkoxy group which may be substituted, and the groups represented by the following formulas: 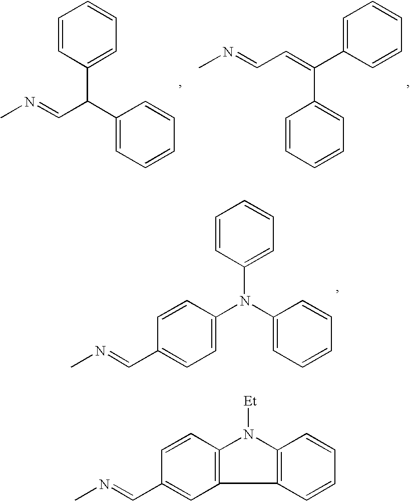
are more preferred. The group represented by the following general formula (II-1): 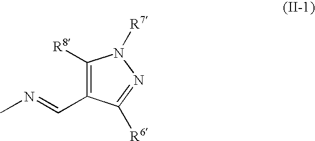
wherein R⁶′ represents a C₁ to C₁₀alkyl group which may be substituted, or a C₆ to C₁₀ aryl group which may be substituted,
R⁷′ represents a C₁ to C₆ alkyl group which may be substituted, or a C₆ to C₁₀ aryl group which may be substituted,
R⁸′ represents a halogen atom, hydroxy group, or a C₁ to C₆ alkoxy group which may be substituted, is further more preferred.

Examples of the substituent, according to “a C₁ to C₁₀alkyl group which may be substituted” and “a C₆ to C₁₀ aryl group which may be substituted” in the definition of R⁶′ include similar groups to the substituents in the expression of “which may be substituted.”

Examples of R⁶′ include a C₁ to C₁₀alkyl group which may be substituted and a C₆ to C₁₀ aryl group which may be substituted. Methyl group, ethyl group, trifluoromethyl group, 2-(ethoxycarbonyl)ethyl group, 2-carboxyethyl group, 2-carbamoylethyl group, 3-(ethoxycarbonyl)propyl group, 3-carboxypropyl group, 3-carbamoylpropyl group, 1-adamantyl group, and 4-tert-butylphenyl group are preferred, and methyl group, trifluoromethyl group, 3-(ethoxycarbonyl)propyl group, and 3-carboxypropyl group are more preferred.

Examples of the substituent, according to “a C₁ to C₆ alkyl group which may be substituted” and “a C₆ to C₁₀ aryl group which may be substituted” in the definition of R⁷′ include similar groups to the substituents in the expression of “which may be substituted.”

Examples of R⁷′ include a C₁ to C₆ alkyl group which may be substituted and a C₆ to C₁₀ aryl group which may be substituted. Methyl group, phenyl group, 2-chlorophenyl group, 3-chlorophenyl group, 4-chlorophenyl group, 4-methylphenyl group, 4-methoxyphenyl group, and 4-nitrophenyl group are preferred, and phenyl group, 3-chlorophenyl group, and 4-nitrophenyl group are more preferred.

Examples of the substituent, according to “a C₁ to C₆ alkoxy group which may be substituted” in the definition of R⁸′ include similar groups to the substituents in the expression of “which may be substituted.”

Examples of R⁸′ include halogen atom, hydroxy group, and a C₁ to C₆ alkoxy group which may be substituted. Halogen atom, hydroxy group, (ethoxycarbonyl)methoxy group, and carboxymethoxy group are preferred, and hydroxy group and carboxymethoxy group are more preferred.

The compounds represented by the following compound group β are excluded from the scope of compounds represented by the aforementioned general formula (I-1).

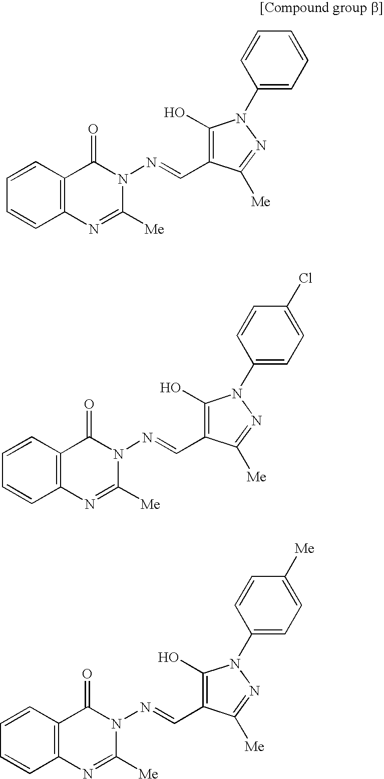

Each compound defined by the aforementioned general formula (I-1) or a pharmacologically acceptable salt thereof, or a hydrate thereof or a solvate thereof is novel, provided that the compounds represented by the aforementioned compound group β are excluded. Uses of the compound according to the aforementioned chemical substance invention are not particularly limited.

The compounds represented by the aforementioned general formulas (I) and (I-1) may form salts. As the pharmacologically acceptable salt, when acidic groups exist, examples include metal salts such as lithium salt, sodium salt, potassium salt, magnesium salt, and calcium salt, or ammonium salts such as ammonium salt, methylammonium salt, dimethylammonium salt, trimethylammonium salt, and dicyclohexylammonium salt, and when basic groups exist, examples include mineral acid salts such as hydrochloride, hydrobromide, sulfate, nitrate, and phosphate, or organic acid salts such as methane sulfonate, benzene sulfonate, para-toluene sulfonate, acetate, propionate, tartrate, fumarate, maleate, malate, oxalate, succinate, citrate, benzoate, mandelate, cinnamate, and lactate. Salts may sometimes be formed with amino acids such as glycine. As active ingredients of the medicaments of the present invention, pharmacologically acceptable salts may also be suitably used.

The compounds or salts thereof represented by the aforementioned general formulas (I) and (I-1) may exist as hydrates or solvates. As an active ingredient of the medicament of the present invention, any of the aforementioned substances may be used. Furthermore, the compounds represented by the general formulas (I) and (I-1) may sometimes have one or more asymmetric carbons, and may exist as stereoisomers such as optically active isomers and diastereomers. As active ingredients of the medicaments of the present invention, a pure form of a stereoisomer, any mixture of enantiomers or diastereomers, a racemate or the like may be used.

Furthermore, in the compounds represented by the general formulas (I) and (I-1), for example, when R is a group represented by the following general formula (II): 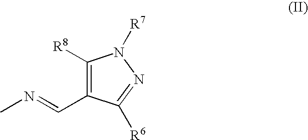
and R⁸ is hydroxy group, or when R′ is a group represented by the following general formula (II-1): 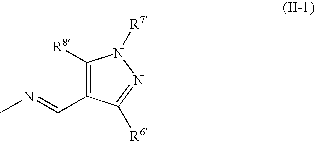
and R⁸′ is hydroxy group, the compounds may exist as pyrazolon form which is a tautomer, in addition to existing as hydroxypyrazole form represented by the aforementioned general formula (II) or (II-1). As active ingredients of the medicament of the present invention, pure forms of tautomers or a mixture thereof may be used. Furthermore, when the compounds represented by the general formulas (I) and (I-1) have double bonds such as olefin, imine and azo, the configuration may be in either E or Z, and as active ingredients of the medicament of the present invention, geometrical isomer in either of the configurations or a mixture thereof may be used.

Examples of the compounds included in the general formulas (I) and (I-1) as active ingredients of the medicaments of the present invention are shown below. However, the active ingredients of the medicaments of the present invention are not limited to the compound set out below.

The abbreviations used in the following tables have the following meanings.

Me: methyl group, Et: ethyl group, i-Pr: isopropyl group, n-Bu: n-butyl group, t-Bu: tert-butyl group, Ph: phenyl group.

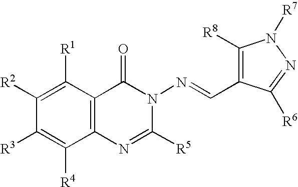

| Compound | | | | | | | | |
|---|---|---|---|---|---|---|---|---|
| Number | R¹ | R² | R³ | R⁴ | R⁵ | R⁶ | R⁷ | R⁸ |
| 1 | H | H | H | H | Me | Me | Ph | OH |
| 2 | H | H | H | H | Me | Me | Ph | Cl |
| 3 | H | H | H | H | Me | Me | Ph | OEt |
| 4 | H | H | H | H | Me | Me | Ph | OCH₂CO₂Et |
| 5 | H | H | H | H | Me | Me | Ph | OCH₂CO₂H |
| 6 | H | H | H | H | Me | Et | Ph | OH |
| 7 | H | H | H | H | Me | CF₃ | Ph | OH |
| 8 | H | H | H | H | Me | (CH₂)₂CO₂Et | Ph | OH |
| 9 | H | H | H | H | Me | (CH₂)₂CO₂H | Ph | OH |
| 10 | H | H | H | H | Me | (CH₂)₂CONH₂ | Ph | OH |
| 11 | H | H | H | H | Me | (CH₂)₃CO₂Et | Ph | OH |
| 12 | H | H | H | H | Me | (CH₂)₃CO₂H | Ph | OH |
| 13 | H | H | H | H | Me | (CH₂)₃CONH₂ | Ph | OH |
| 14 | H | H | H | H | Me | Me | Me | OH |
| 15 | H | H | H | H | Me | Me | 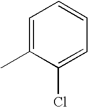 | OH |
| 16 | H | H | H | H | Me | Me | 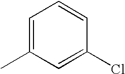 | OH |
| 17 | H | H | H | H | Me | Me | 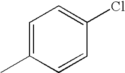 | OH |
| 18 | H | H | H | H | Me | Me | 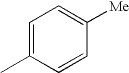 | OH |
| 19 | H | H | H | H | Me | Me | 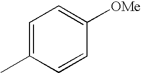 | OH |
| 20 | H | H | H | H | Me | Me | 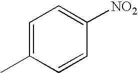 | OH |
| 21 | H | H | H | H | Et | Me | Ph | OH |
| 22 | H | H | H | H | i-Pr | Me | Ph | OH |
| 23 | H | H | H | H | n-Bu | Me | Ph | OH |
| 24 | H | H | H | H | Ph | Me | Ph | OH |
| 25 | H | H | H | H | t-Bu | Me | Ph | OH |
| 26 | H | H | H | H | CH₂OH | Me | Ph | OH |
| 27 | H | H | H | H | CH₂OH | Me | Ph | OCH₂CO₂Et |
| 28 | H | H | H | H | CH₂OH | CF₃ | Ph | OH |
| 29 | H | H | H | H | CH₂OMe | Me | Ph | OH |
| 30 | H | H | H | H | 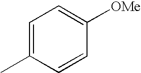 | Me | Ph | OH |
| 31 | H | H | H | H | 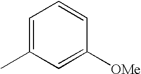 | Me | Ph | OH |
| 32 | H | H | H | H | 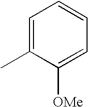 | Me | Ph | OH |
| 33 | Me | H | H | H | Me | Me | Ph | OH |
| 34 | H | H | H | Me | Me | Me | Ph | OH |
| 35 | H | Cl | H | H | Me | Me | Ph | OH |
| 36 | H | H | Cl | H | Me | Me | Ph | OH |
| 37 | H | Br | H | H | Me | Me | Ph | OH |
| 38 | H | OMe | OMe | H | Me | Me | Ph | OH |
| 44 | H | H | H | H | Me | 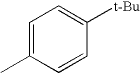 | Ph | OH |
| 45 | H | H | H | H | Me | 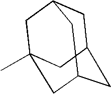 | Ph | OH |

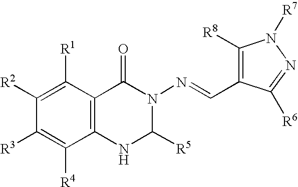

| Compound | | | | | | | | |
|---|---|---|---|---|---|---|---|---|
| Number | R¹ | R² | R³ | R⁴ | R⁵ | R⁶ | R⁷ | R⁸ |
| 39(*) | H | H | H | H | CH₂OH | Me | Ph | OH |
| 40(**) | H | H | H | H | CH₂OH | Me | Ph | OH |
| 41(***) | H | H | H | H | CH₂OH | Me | Ph | OH |

(*)racemate
(**)optically active form
(***)enantiomer of compound No.40

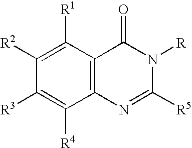

| Compound | | | | | | |
|---|---|---|---|---|---|---|
| Number | R¹ | R² | R³ | R⁴ | R⁵ | R |
| 42 | H | H | H | H | Me | 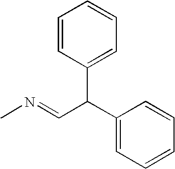 |
| 43 | H | H | H | H | Me | 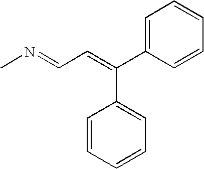 |
| 46 | H | H | H | H | Me | 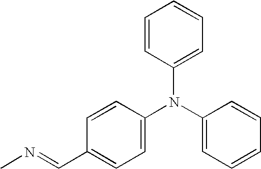 |
| 47 | H | H | H | H | Me | 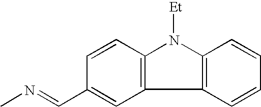 |

The compounds represented by the general formulas (I) and (I-1) can be prepared, for example, by methods shown below.

[1] Method for the Preparation of the Compounds wherein R or R′ is a C₁ to C₆ Alkylideneamino Group which may be Substituted.

The compounds wherein R or R′ is a C₁ to C₆ alkylideneamino group which may be substituted can be prepared, for example, by dehydrocondensation of a 3-amino-3,4-dihydroquinazolin-4-one derivative or a 3-amino-1,2,3,4-tetrahydroxyquinalolin-4-one derivative and an aldehyde derivative or an ketone derivative. The aldehyde derivatives and the ketone derivatives can be prepared by commercially available compounds or by preparation methods described in various kinds of well-known literatures.

As typical examples, methods for the preparation of the compounds wherein R is the following general formula (II): 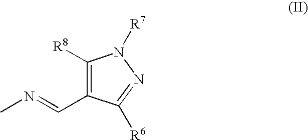
and the compounds wherein R′ is the following general formula (II-1): 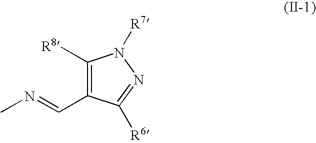
are shown below which are preferred embodiments of the present invention.
(1) Preparation of the Pyrazole Part

The pyrazole part can be prepared, for example, by a method described in the following reaction scheme 1 as an aldehyde derivative. 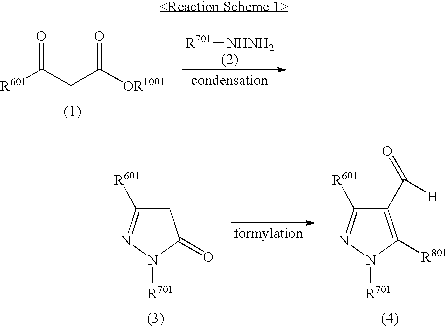
wherein R⁶⁰¹ represents R⁶ in the general formula (II) or its precursor, R⁷⁰¹ represents R⁷ in the general formula (II) or its precursor, R⁸⁰¹ represents R⁸ in the general formula (II) or its precursor, R¹⁰⁰¹ represents an alkyl group such as methyl group and ethyl group, or an aralkyl group such as benzyl group.

The 2,5-disubstituted-2,4-dihydropyrazol-3-one derivative (3) can be prepared, for example, by dehydrocondensation of the β-ketoester derivative (1) and the hydrazine derivative (2). This reaction is carried out at a reaction temperature of from 0° C. to the boiling point of the solvent, without solvent or in a solvent.

As the solvent, any solvent can be used as long as it does not inhibit the reaction, and examples include, for example, alcohols such as methanol, ethanol, 2-propanol, ethyleneglycol monomethyl ether and ethyleneglycol; ethers such as tetrahydrofuran, 1,4-dioxane and 1,2-dimethoxyethane; and aromatic solvents such as benzene, toluene, monochlorobenzene, o-dichlorobenzene and xylene. These solvents may be used alone or as a mixture of two or more kinds of solvents.

Then, the 1,3,5-trisubstituted-4-formyl-1H-pyrazole derivative (4) can be prepared by formylation of the resulting 2,5-disubstituted-2,4-dihydropyrazol-3-one derivative (3). This reaction is carried out at a reaction temperature of from 0° C. to the boiling point of the solvent, preferably, at a reaction temperature of from 60° C. to 100° C., in N,N-dimethylformamide, in the presence of phosphorus oxychloride. After the reaction is completed, when the reaction mixture is treated with water, a compound wherein R⁸⁰¹ is hydroxy group can be obtained. When the reaction mixture is treated with sodium hydrogencarbonate solution, a compound wherein R⁸⁰¹ is chloro group can be obtained. Then, functional group conversion reactions such as alkylation (whose examples include, for example, (ethoxycarbonyl)methylation using bromoacetic acid ethyl ester) for the compounds wherein R⁸⁰¹ is hydroxy group; and substitution reaction (whose examples include, for example, introduction of ethoxy group by a reaction with sodium ethoxide) for the compounds wherein R⁸⁰¹ is hydroxy group. Various well-known functional group conversion reactions can be used for said functional group conversion reactions, and, for example, methods described in “Protective Groups in Organic Syntheses”, (USA), Theodra W. Green, Peter G. M. Wuts, Eds., Third edition, April in 1999, John Wiley & Sons, and “Handbook of Reagents for Organic Synthesis”, (USA), 4 Volumes, June in 1999, John Wiley & Sons can be used.

(2) Preparation of the Quinazoline Part

The quinazoline part can be prepared, for example, by a method described in the following reaction scheme 2 as a 3-amino-3,4-dihydroquinazolin-4-one derivative or a 3-amino-1,2,3,4-tetrahydroquinazolin-4-one derivative. 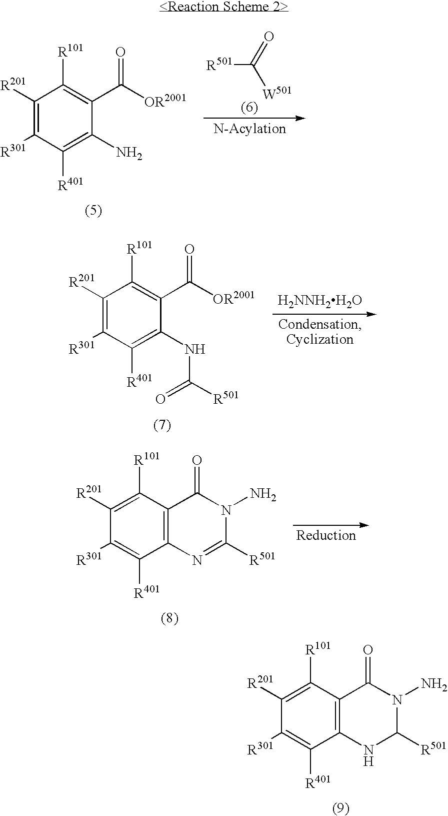
wherein R¹⁰¹ represents R¹ in the general formula (I) or its precursor, R²⁰¹ represents R² in the general formula (I) or R²′ in the general formula (I-1), or a precursor thereof, R³⁰¹ represents R³ in the general formula (I) or its precursor, R⁴⁰¹ represents R⁴ in the general formula (I) or its precursor, R⁵⁰¹ represents R⁵ in the general formula (I) or its precursor, R²⁰⁰¹ represents an alkyl group such as methyl group and ethyl group, or an aralkyl group such as benzyl group, and W⁵⁰¹ represents halogen atoms and the like.

The 2-(acylamino) benzoate derivative (7) can be prepared, for example, by N-acylation of the anthranilic acid ester derivative (5) with the acylating agent (6).

This reaction is carried out at a reaction temperature of from −80° C. to the boiling point of the solvent, without solvent or in a solvent, in the presence or absence of a base and/or a catalyst. As the base, examples include inorganic bases such as sodium carbonate, potassium carbonate, and sodium hydrogencarbonate; and organic bases such as pyridine, triethylamine, and N,N-diethylaniline. As the catalyst, examples include mineral acids such as hydrochloric acid and sulfuric acid; organic acids such as acetic acid, methanesulfonic acid, para-toluenesulfonic acid; and organic bases such as 4-dimethylaminopyridine and diisopropylethylamine. As the solvent, any solvent can be used as long as it does not inhibit the reaction, and examples include, for example, halogenated solvents such as dichloromethane, dichloroethane, and chloroform; ethers such as tetrahydrofuran, 1,4-dioxane and 1,2-dimethoxyethane; and aromatic solvents such as benzene, toluene, monochlorobenzene, o-dichlorobenzene and xylene; and amides such as N,N-dimethylformamide and N-methylpyrrolidone. These solvents may be used alone or as a mixture of two or more kinds of solvents.

Then, the 3-amino-3,4-dihydroquinazolin-4-one derivative (8) can be prepared by condensation and cyclization of the obtained 2-(acylamino)benzoate derivative (7) and hydrazine mono hydrate. This reaction is carried out at the reaction temperature of from 0° C. to the boiling point of the solvent, without solvent or in a solvent.

As the solvent, any solvent can be used as long as it does not inhibit the reaction, and examples include, for example, alcohols such as methanol, ethanol, 2-propanol, ethyleneglycol monomethyl ether, and ethylene glycol; ethers such as tetrahydrofuran, 1,4-dioxane, and 1,2-dimethoxyethane; and aromatic solvents such as benzene, toluene, monochlorobenzene, o-dichlorobenzene, and xylene. These solvents may be used alone or as a mixture of two or more kinds of solvents.

Then, the 3-amino-1,2,3,4-tetrahydroquinazolin-4 one derivative (9) can be prepared by reduction of the obtained 3-amino-3,4-dihydroquinazolin-4-one derivative (8). This reaction is carried out, for example, at the reaction temperature of from 0° C. to the boiling point of the solvent, in a solvent, in the presence of catalyst, under hydrogen atmosphere. As the catalyst, examples include rare metal catalysts such as palladium carbon and palladium black. As the solvent, any solvent can be used as long as it does not inhibit the reaction, and examples include, for example, alcohols such as methanol, ethanol, 2-propanol, ethyleneglycol monomethyl ether, and ethylene glycol; ethers such as tetrahydrofuran, 1,4-dioxane, and 1,2-dimethoxyethane; and water. These solvents may be used alone or as a mixture of two or more kinds of solvents.

Furthermore, the 3-amino-3,4-dihydroquinazolin-4-one derivative (8) can also be prepared by a method described in the following reaction scheme 3. 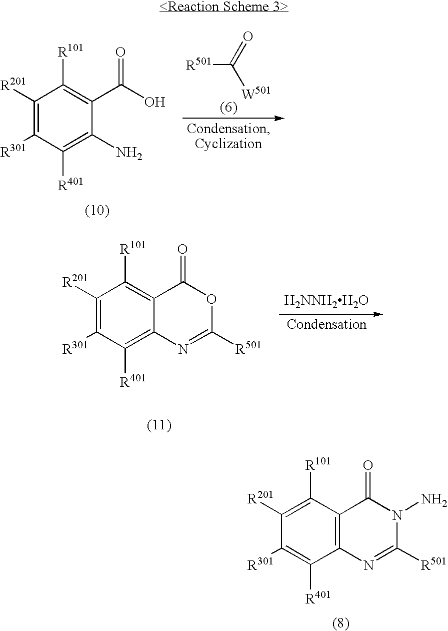
wherein R¹⁰¹ represents R¹ in the general formula (I) or its precursor, R²⁰¹ represents R² in the general formula (I) or its precursor, R³⁰¹ represents R³ in the general formula (I) or its precursor, R⁴⁰¹ represents R⁴ in the general formula (I) or its precursor, R⁵⁰¹ represents R⁵ in the general formula (I) or its precursor, and W⁵⁰¹ represents halogen atoms and the like.

The 4H-3,1-benzoxazin-4-one derivative (11) can be prepared by condensation and cyclization of the anthranilic acid derivative (10) and the acylating agent (6). This reaction is carried out at the reaction temperature of from −80° C. to the boiling point of the solvent without solvent or in a solvent, in the presence or absence of a base. As the base, examples include inorganic bases such as sodium carbonate, potassium carbonate, and sodium hydrocarbonate; and organic bases such as pyridine, triethylamine, and N,N-diethylaniline. As the solvent, any solvent can be used as long as it does not inhibit the reaction, and examples include, for example, halogenated solvents such as dichloromethane, dichloroethane, and chloroform; ethers such as tetrahydrofuran, 1,4-dioxane and 1,2-dimethoxyethane; and aromatic solvents such as benzene, toluene, monochlorobenzene, o-dichlorobenzene and xylene; and amides such as N,N-dimethylformamide and N-methylpyrrolidone. These solvents may be used alone or as a mixture of two or more kinds of solvents. Furthermore, bases such as pyridine may be used as a solvent.

Then, the 3-amino-3,4-dihydroquinazolin-4-one derivative (8) can be prepared by condensation of the obtained 4H-3,1-benzoxazin-4-one derivative (11) and the hydrazine mono hydrate. This reaction is carried out at the reaction temperature of from 0° C. to the boiling point of the solvent, without solvent or in a solvent. As the solvent, any solvent can be used as long as it does not inhibit the reaction, and examples include, for example, alcohols such as methanol, ethanol, 2-propanol, ethyleneglycol monomethyl ether, and ethylene glycol; ethers such as tetrahydrofuran, 1,4-dioxane, and 1,2-dimethoxyethane; and aromatic solvents such as benzene, toluene, monochlorobenzene, o-dichlorobenzene, and xylene. These solvents may be used alone or as a mixture of two or more kinds of solvents.

(3) Preparation of the Compounds Represented by the General Formula (1) by Condensation of the Pyrazole Part and the Quinazoline Part, and Functional Group Conversion.

The compounds represented by the general formula (1) can be prepared, for example, by methods described in the following reaction scheme 4 and reaction scheme 5. 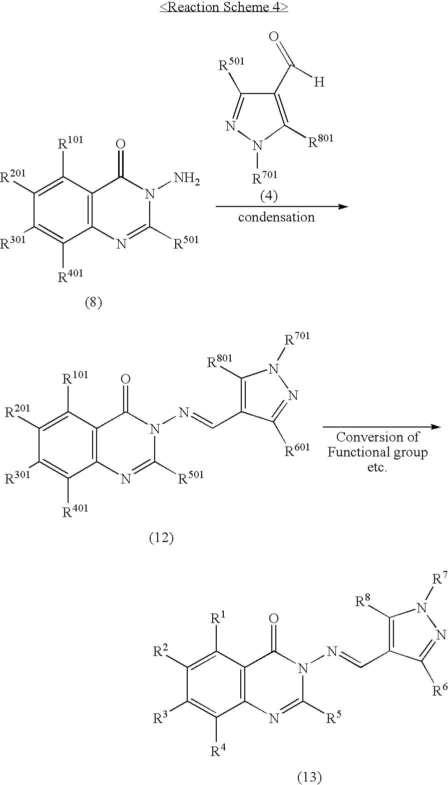
wherein each of R¹, R², R³, R⁴, R⁵, R⁶, R⁷, and R⁸ has the same meaning as those defined in the general formulas (I) and (II), R¹⁰¹ represents R¹ in the general formula (I) or its precursor, R²⁰¹ represents R² in the general formula (I) or its precursor, R³⁰¹ represents R³ in the general formula (I) or its precursor, R⁴⁰¹ represents R⁴ in the general formula (I) or its precursor, R⁵⁰¹ represents R⁵ in the general formula (I) or its precursor, R⁶⁰¹ represents R⁶ in the general formula (II) or its precursor, R⁷⁰¹ represents R⁷ in the general formula (II) or its precursor, R⁸⁰¹ represents RS in the general formula (II) or its precursor. 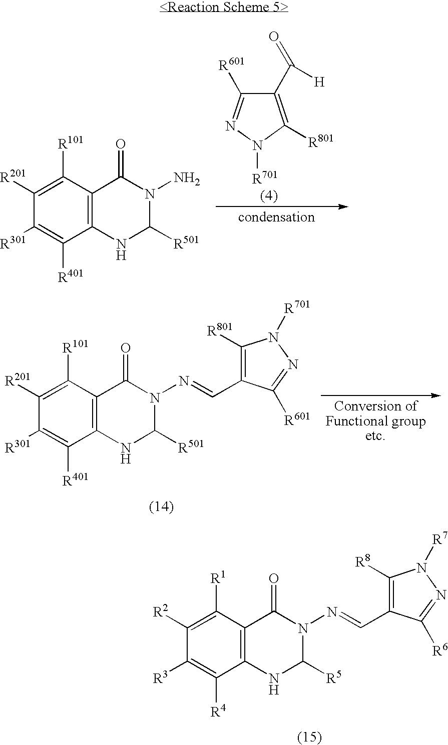
wherein each of R¹, R², R³, R⁴, R⁵, R⁶, R⁷, and R⁸ has the same meaning as those defined in the general formulas (I) and (II), R₁₀₁ represents R¹ in the general formula (I) or its precursor, R²⁰¹ represents R² in the general formula (I) or its precursor, R³⁰¹ represents R³ in the general formula (I) or its precursor, R⁴⁰¹ represents R⁴ in the general formula (I) or its precursor, R⁵⁰¹ represents R⁵ in the general formula (I) or its precursor, R⁶⁰¹ represents R⁶ in the general formula (II) or its precursor, R⁷⁰¹ represents R⁷ in the general formula (II) or its precursor, R⁸⁰¹ represents R⁸ in the general formula (II) or its precursor.

The imine derivative (12) can be prepared, for example, by dehydrocondensation of the 3-amino-3,4-dihydroquinazolin-4-one derivative (8) and the 1,3,5-trisubstituted-4-formyl-1H-pyrazole derivative (4). This reaction is carried out at a reaction temperature of from 0° C. to the boiling point of the solvent, without solvent or in a solvent, in the presence or absence of a catalyst. As the catalyst, examples include mineral acids such as hydrochloric acid and sulfuric acid; organic acids such as acetic acid, methanesulfonic acid, para-toluenesulfonic acid; and organic bases such as 4-dimethylaminopyridine and diisopropylethylamine. As the solvent, any solvent can be used as long as it does not inhibit the reaction, and examples include, for example, alcohols such as methanol, ethanol, 2-propanol, ethyleneglycol monomethyl ether, and ethylene glycol; halogenated solvents such as dichloromethane, dichloroethane, and chloroform; ethers such as tetrahydrofuran, 1,4-dioxane, and 1,2-dimethoxyethane; aromatic solvents such as benzene, toluene, monochlorobenzene, o-dichlorobenzene, and xylene; amides such as N,N-dimethylformamide and N-methylpyrrolidone; and acetic acid. These solvents may be used alone or as a mixture of two or more kinds of solvents.

When the functional group conversion or the like of the imine derivative (12) needs to be carried out, the final target compound (13) can be prepared by carrying out the functional group conversion reaction at the end. Various well-known functional group conversion reactions can be used for said functional group conversion reactions, and, for example, methods described in “Protective Groups in Organic Syntheses”, (USA), Theodra W. Green, Peter G. M. Wuts, Eds., Third edition, April in 1999, John Wiley & Sons, and “Handbook of Reagents for Organic Synthesis”, (USA), 4 Volumes, June in 1999, John Wiley & Sons can be used.

Similarly, the imine derivative (14) and the final target compound (15) can be prepared by using the 3-amino-1,2,3,4-tetrahydroquinazolin-4-one derivative (9) and the 1,3,5-trisubstituted-4-formyl-1H-pyrazole derivative (4).

The compounds represented by the general formula (1-1) can also be prepared in the same manner.

[2] Method for the Preparation Method of the Compounds Wherein R or R′ is an Amino Group which may be Substituted.

The compounds wherein R or R′ is an amino group which may be substituted can be prepared, for example, by a method described in the following reaction scheme 6, which is dehydrocondensation of the 4H-3,1-benzoxazin-4-one derivative and the hydrazine derivative. 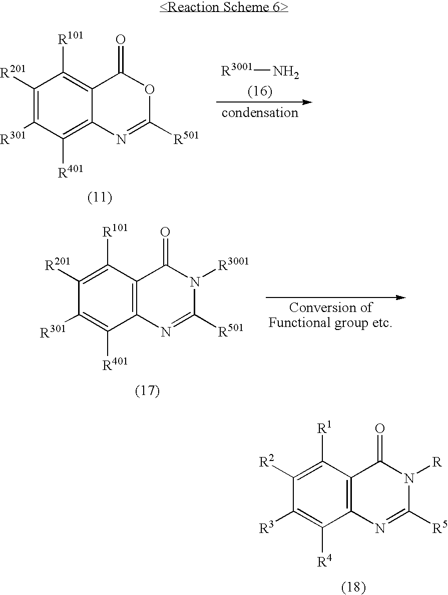
wherein each of R¹, R², R³, R⁴, and R⁵ has the same meaning as those defined in the general formula (I), R¹⁰¹ represents R¹ in the general formula (I) or its precursor, R²⁰¹ represents R² in the general formula (I) or its precursor, R³⁰¹ represents R³ in the general formula (I) or its precursor, R⁴⁰¹ represents R⁴ in the general formula (I) or its precursor, R⁵⁰¹ represents R⁵ in the general formula (I) or its precursor, R represents an amino group which may be substituted, and R³⁰⁰¹ represents an amino group which may be substituted or its precursor.

The 3,4-dihydroquinazolin-4-one derivative (17) can be prepared by dehydrocondensation of the 4H-3,1-benzoxazin-4-one derivative (11) and the hydrazine derivative (16).

This reaction is carried out at a reaction temperature of from 0° C. to the boiling point of the solvent, without solvent or in a solvent, in the presence or absence of a catalyst. As the catalyst, examples include mineral acids such as hydrochloric acid and sulfuric acid; organic acids such as acetic acid, methanesulfonic acid, para-toluenesulfonic acid; and organic bases such as 4-dimethylaminopyridine and diisopropylethylamine. As the solvent, any solvent can be used as long as it does not inhibit the reaction, and examples include, for example, alcohols such as methanol, ethanol, 2-propanol, ethyleneglycol monomethyl ether, and ethylene glycol; halogenated solvents such as dichloromethane, dichloroethane, and chloroform; ethers such as tetrahydrofuran, 1,4-dioxane, and 1,2-dimethoxyethane; aromatic solvents such as benzene, toluene, monochlorobenzene, o-dichlorobenzene, and xylene; amides such as N,N-dimethylformamide and N-methylpyrrolidone; and acetic acid. These solvents may be used alone or as a mixture of two or more kinds of solvents.

When the functional group conversion or the like of the 3,4-dihydroquinazolin-4-one derivative (17) needs to be carried out, the final target compound (18) can be prepared by carrying out the functional group conversion reaction at the end. Various well-known functional group conversion reactions can be used for said functional group conversion reactions, and, for example, methods described in “Protective Groups in Organic Syntheses”, (USA), Theodra W. Green, Peter G. M. Wuts, Eds., Third edition, April in 1999, John Wiley & Sons, and “Handbook of Reagents for Organic Synthesis”, (USA), 4 Volumes, June in 1999, John Wiley & Sons can be used.

The hydrazine derivative (16) can be prepared by commercially available compounds or by preparation methods described in various kinds of well-known literatures.

When the compounds represented by the general formula (I) have one or more asymmetric carbons and their optically active substances are to be prepared, each of a method using optically active raw materials, and a method wherein the racemate is first prepared, and then optical resolution is carried out may be used. As the method for the optical resolution, various well-known methods by those skilled in the art, for example, a method using a high performance liquid chromatography with an optically active column can be used.

The compounds represented by the general formula (I-1) can also be prepared by the same manner. The compounds represented by the general formulas (I) and (I-1) prepared by the aforementioned methods can be isolated and purified by methods widely known by those skilled in the art, for example, extraction, precipitation, fractional chromatography, fractional crystallization, suspension and washing, and recrystallization. Furthermore, each of the pharmaceutically acceptable salt of the compound of the present invention, the hydrate thereof and the solvate thereof can be prepared by methods widely known by those skilled in the art.

In the examples of the specification, preparation methods of typical compounds included in the general formulas (I) and (I-1) are explained in details. Therefore, those skilled in the art can prepare any compound fall within the general formulas (I) and (I-1) by referring to the explanations of the aforementioned general preparation methods and those of specific preparation methods of the examples, by choosing appropriate reaction raw materials, reaction reagents, and reaction conditions, and by adding appropriate modification and alteration of these methods, if necessary.

The compounds represented by the general formulas (I) and (I-1) have inhibitory activity against hematopoietic prostaglandin D2(PGD2) synthase, and they can preferably be used as an allergic inflammation inhibitor. The aforementioned medicament is useful as an active ingredient of a medicament preventive and/or therapeutic treatment of inflammatory diseases caused by an allergic reaction. More specifically, the medicament of the present invention may be used for preventive and/or therapeutic treatment of the following diseases wherein an allergic inflammation reaction is believed to be involved, for example, allergic diseases such as contact dermatitis, atopic dermatitis, eczema, pollinosis, asthma, bronchitis, angitis, rhinitis, nasal obstruction, intestinal pneumonia, arthritis, opthalmia, conjunctivitis, neuritides, middle otitis, encephalomyelitis, cystitis, adenoiditis, food allergy, insect allergy, drug allergy, and anaphylactic shock, moreover, for the enlargement of tissue damage involving vasodilation, vascular permeability, and infiltration of inflammatory cell due to the overproduction of prostaglandin D2.

Furthermore, from the recent studies, in the brain damaged site by diseases such as cerebrovascular damage, brain degenerative disease, and demyelinating disease, it was confirmed that the expression of hematopoietic prostaglandin D2 synthase (H-PGDS) increases and in the astroglia cells which is remarkably activated in the damaged site, prostaglandin D receptor (DP receptor) is induced (the specification of Japanese Patent Application (TOKUGAN) No. 2002-204725). When a H-PDGS inhibitor, like HQL-79, or a DP receptor antagonist is administered to brain damage model animals, the activation of the astroglia cells is inhibited, and the damage is aggravated in the brain damage model in the H-PGDS mass expression transgenic mouse, consequently, it is obvious that PGD2 is related to the aggravation of the brain damage. Therefore, a strong H-PGDS inhibitor is useful as a medicament for preventing the aggravation of brain damage, and/or improving the prognosis of brain damage, and the medicament of the present invention can be used for this purpose. Kind of brain damages that are applicable targets for the medicament of the present invention are not particularly limited. Examples include, for example, those traumatic by traffic accidents, those by cerebrovascular damage such as stroke and brain hemorrhage, those by brain degenerative diseases, demyelinatng diseases, however, the diseases are not limited to these examples.

Furthermore, since prostaglandin D2 is known to be involved in induction of sleep, other reduction of body temperature, inhibition of progestin secretion, and response regulatory action of pain and smell (Vitamins and hormones, (USA), 2000, Vol. 58, p. 89-120; The Journal of Biological Chemistry, (USA), 1985, Vol. 260, No. 23, p. 12140-12145; Biochemica et Biophysica Acta, (Netherlands), 2000, Vol. 1482, No. 1-2, p. 259-271), the medicament of the present invention is useful as a medicament having one or more activities selected from the group consisting of an estrous cycle regulatory activity, sleep regulatory activity, thermoregulatory activity, analgesic activity and olfaction regulatory activity.

As the active ingredient of the medicament on the present invention, one or more kinds of substances selected from the group consisting of the compound represented by the general formulas (I) and (I-1), and a pharmacologically acceptable salt thereof, and a hydrate thereof and a solvate thereof may be used. The aforementioned substance, per se, may be administered as the medicament of the present invention, however, preferably, the medicament of the present invention is provided in the form of a pharmaceutical composition comprising the aforementioned substance which is an active ingredient together with one or more pharmacologically acceptable pharmaceutical additives. In the aforementioned pharmaceutical compositions, a ratio of the active ingredient to the pharmaceutical additives is 1 weight % to 90 weight %.

The pharmaceutical compositions of the present invention may be administered as pharmaceutical compositions for oral administration, for example, granules, subtilized granules, powders, hard capsules, soft capsules, syrup, emulsion, suspension, or solution, or may be administered as pharmaceutical compositions for parenteral administration, for example, injections for intravenous administration, intramuscular administration, or subcutaneous administration, drip infusions, suppositories, percutaneous absorbent, transmucosal absorption preparations, nasal drops, ear drops, instillation, and inhalants. Preparations made as pharmaceutical compositions in a form of powder may be dissolved when necessary and used as injections or drip infusions.

For preparation of pharmaceutical compositions, solid or liquid pharmaceutical additives may be used. Pharmaceutical additives may either be organic or inorganic. When an oral solid preparation is prepared, an excipient is added to the active ingredient, and further binders, disintegrator, lubricant, colorant, corrigent are added, if necessary, to manufacture preparations in the forms of tablets, coating tablets, granules, powders, capsules and the like by ordinary procedures. Examples of the excipient include lactose, sucrose, saccharose, glucose, corn starch, starch, talc, sorbit, crystal cellulose, dextrin, kaolin, calcium carbonate, and silicon dioxide. Examples of the binder include, for example, polyvinyl alcohol, polyvinyl ether, ethyl cellulose, methyl cellulose, gum Arabic, tragacanth, gelatine, shellac, hydroxypropyl cellulose, hydroxypropyl methyl cellulose, calcium citrate, dextrin, and pectin. Examples of the lubricant include, for example, magnesium stearate, talc, polyethylene glycol, silica, and hydrogenated vegetable oil. As the coloring agent, any material can be used which are approved to be added to ordinary pharmaceuticals. As the corrigent, cocoa powder, menthol, aromatic acid, peppermint oil, d-borneol, cinnamon powder and the like can be used. These tables and granules may be applied with sugarcoating, gelatin coating, or an appropriate coating, if necessary. Preservatives, antioxidant and the like may be added, if required.

For liquid preparations for oral administration such as emulsions, syrups, suspensions, and solutions, ordinary used inactive diluents, for example, water or vegetable oil may be used. For these preparations, besides inactive diluents, adjuvants such as wetting agents, suspending aids, sweating agents, flavoring agents, coloring agents or preservatives may be blended. After a liquid preparation is manufactured, the preparation may be filled in capsules made of a absorbable substance such as gelatin. Examples of solvents or suspending agents used for the preparations of parenteral administration such as injections or suppositories include, for example, water, propylene glycol, polyethylene glycol, benzyl alcohol, ethyl oleate, and lecithin. Examples of base materials used for preparation of suppositories include, for example, cacao butter, emulsified cacao butter, lauric fat, and witepsol. Methods for preparation of the aforementioned preparations are not limited, and any method ordinarily used in the art may be used.

When the composition are prepared in the form of injections, carriers such as, for example, diluents including water, ethanol, macrogol, propylene glycol, citric acid, acetic acid, phosphoric acid, lactic acid, sodium lactate, sulfuric acid and sodium hydroxide, pH modifiers and buffer solutions including sodium citrate, sodium acetate and sodium phosphate, stabilizers such as sodium pyrosulfite, ethylenediaminetetraacetic acid, thioglycolic acid and thiolactate may be used. For the preparation, a sufficient amount of a salt, glucose, mannitol or glycerin may be blended in the preparation to manufacture an isotonic solution, and an ordinary solubilizer, a soothing agent, or a topical anesthetic may be used.

When the preparation in the form of an ointment such as a paste, a cream, and a gel is manufactured, an ordinarily used base material, a stabilizer, a wetting agent, and a preservative may be blended, if necessary, and may be prepared by mixing the components by a common method. As the base material, for example, white petrolatum, polyethylene, paraffin, glycerin, cellulose derivatives, polyethylene glycol, silicon, and bentonite may be used. As the preservative, paraoxy methyl benzoate, paraoxy ethyl benzoate, paraoxy propyl benzoate and the like may be used. When the preparation in the form of a patch is manufactured, the aforementioned ointment, cream gel, or paste and the like may be applied by a common method to an ordinary support. As the support, fabric made of cotton, span rayon, and synthetic fibersor or nonwoven fabric, and a film or a foam sheet such as made of soft vinyl chloride, polyethylene, and polyurethane and the like may be preferably used.

A dose of the medicament of the present invention is not particularly limited. For oral administration, a dose may generally be 0.01 to 5,000 mg per day for an adult as the weight of the compound of the present invention. It is preferred to increase or decrease the above dose appropriately depending on the age, pathological conditions, and symptoms of a patient. The above dose may be administered once a day or 2 to 3 times a day as divided portions with appropriate intervals, or intermittent administration for every several days may be applied. When the medicament is used as an injection, the dose may be 0.001 to 100 mg per day for an adult as the weight of the compound of the present invention.

### EXAMPLES

The present invention will be explained more specifically with reference to the following examples. However the scope of the present invention is not limited to the following examples. The compound numbers in the following examples correspond to those in the table shown above.

### Example 1

### Preparation of the Compound of Compound No. 1

### (1) 2-(Acetamide)benzoic acid methyl ester

Acetyl chloride (1.57 mL, 22 mmol) was added to a solution of anthranilic acid methyl ester (3.02 g, 20 mmol) and triethylamine (3.5 mL, 25 mmol) in dichloromethane (100 mL) under ice cooling, and the mixture was stirred at room temperature overnight. The reaction mixture was poured into ice and water, and extracted with dichloromethane. The dichloromethane layer was washed with brine and dried over anhydrous sodium sulfate. The solid obtained by evaporation of the solvent under reduced pressure was washed with methanol under suspension and recrystallized from n-hexane/ethyl acetate to give the title compound (0.98 g, 25.4%) as a white crystal.

¹H-NMR(CDCl₃): δ 2.72(3H, s), 4.90(2H, s), 7.45(1H, t, J=6.9 Hz), 7.63-7.71(1H, m), 7.74-7.77(1H, m), 8.22-8.25(1H, m).

### (2) 3-Amino-2-methyl-3,4-dihydroquinazolin-4-one

Hydrazine monohydrate (1.4 mL, 28.9 mmol) was added to a solution of 2-(acetamido)benzoic acid methyl ester (0.95 g, 4.92 mmol) in ethanol (5 mL), and the mixture was refluxed for 8 hours. After the reaction mixture was cooled to room temperature, the separated crystal was filtered and recrystallized from methanol to give the title compound (0.42 g, 48.7%) as a white crystal.

¹H-NMR(CDCl₃): δ 2.72(3H, s), 4.90(2H, s), 7.45(1H, t, J=6.9 Hz), 7.63-7.71(1H, m), 7.74-7.77(1H, m), 8.22-8.25(1H, m).

### (3) 3-Methyl-5-oxo-1-phenyl-4,5-dihydropyrazole-4-carbaldehyde

Phosphorus oxychloride (1.85 mL, 19.8 mmol) was added to a solution of 3-methyl-1-phenyl-4,5-dihydropyrazol-5-one (2.90 g, 16.6 mmol) in N,N-dimethylformamide (4.0 mL) under ice cooling, and the mixture was stirred at 80° C. for 1 hour. After the reaction mixture was cooled to room temperature, it was poured into ice and water, and stirred at room temperature overnight. The separated solid was filtered and dried under reduced pressure. The solid was washed with isopropyl ether to give the title compound (1.70 g, 50.4%) as an yellow solid.

¹H-NMR(CDCl₃): δ 2.43(3H, s), 7.28-7.33(1H, m), 7.42-7.48(2H, m), 7.79-7.82(2H, m), 9.52(1H, s), 9.90(1H, s).

### (4) Preparation of 3-[(5-hydroxy-3-methyl-1-phenylpyrazol-4-yl)methylidene]-amino-2-methyl-3,4-dihydroquinazolin-4-one (Compound No. 1)

3-Amino-2-methyl-3,4-dihydroquinazolin-4-one (0.05 g, 0.285 mmol) was added to a solution of 3-methyl-5-oxo-1-phenyl-4,5-dihydropyrazole-4-carbaldehyde (0.069 g, 0.342 mmol) in ethanol (1 mL), and the mixture was stirred at room temperature for 2 hours. The separated solid was filtered and washed with ethanol to give the title compound (0.084 g, 81.6%) as an yellow crystal.

¹H-NMR(CDCl₃): δ 2.25(3H, s), 2.64(3H, s), 7.14(1H, t, J=7.5 Hz), 7.35(2H, t, J=8.1 Hz), 7.47-7.53(2H, m), 7.65(1H, d, J=7.8 Hz), 7.77-7.82(1H, m), 7.88(2H, dd, J=8.7, 1.2 Hz), 8.23(1H, dd, J=7.8, 1.2 Hz).

### Example 2

### Preparation of the Compound of Compound No. 2

### (1) Preparation of 5-chloro-3-methyl-1-phenylpyrazole-4-carbaldehyde

Phosphorus oxychloride (1.12 mL, 12 mmol) was added to a solution of 3-methyl-1-phenyl-4,5-dihydropyrazol-5-one (1.74 g, 10 mmol) in N,N-dimethylformamide (2 mL) under ice cooling, and the mixture was stirred at 80° C. for 1 hour. After the reaction mixture was cooled to room temperature, it was poured into ice and water. Aqueous sodium hydrogen carbonate was added to the mixture and it was stirred at room temperature overnight. The mixture was extracted with ethyl acetate, and the ethyl acetate layer was washed with brine and dried over anhydrous sodium sulfate. The residue obtained by evaporation of the solvent under reduced pressure was purified by column chromatography on silica gel (chloroform) to give the title compound (1.237 g, 56.1%) as a white solid.

¹H-NMR(CDCl₃): δ 2.53(3H, s), 7.46-7.54(5H, m), 9.96(1H, s).

### (2) Preparation of 3-[(5-chloro-3-methyl-1-phenylpyrazol-4-yl)methylidene]-amino-2-methyl-3,4-dihydroquinazolin-4-one (Compound No. 2)

3-Amino-2-methyl-3,4-dihydroquinazolin-4-one (compound of Example 1(2); 17.5 mg, 0.1 mmol) and catalytic amount of p-toluenesulfonic acid were added to a solution of 5-chloro-3-methyl-1-phenylpyrazole-4-carbaldehyde (22 mg, 0.1 mmol) in toluene (2.5 mL), and the mixture was refluxed for 8 hours. After the reaction mixture was cooled to room temperature, the insoluble matter was filtered off, and the filtrate was washed with saturated aqueous sodium hydrogen carbonate and dried over anhydrous sodium sulfate. The residue obtained by evaporation of the solvent under reduced pressure was purified by preparative thin layer chromatography on silica gel (chloroform) to give the title compound (14.4 mg, 38.1%) as a light yellow solid.

¹H-NMR(DMSO-d₆): δ 2.50(3H, s), 2.55(3H, s), 7.50-7.67(7H, m), 7.83(1H, t, J=7.0 Hz), 8.17(1H, d, J=7.7 Hz), 8.90(1H, s).

### Example 3

### Preparation of the Compound of Compound No. 3

### (1) Preparation of 5-ethoxy-3-methyl-1-phenylpyrazole-4-carbaldehyde

Sodium ethoxide (0.044 g, 0.65 mmol) was added to a solution of 5-chloro-3-methyl-1-phenylpyrazole-4-carbaldehyde (compound of Example 2(1); 0.110 g, 0.5 mmol) in ethanol (4 mL), and the mixture was stirred at 60° C. for 5 hours. After the reaction mixture was cooled to room temperature, water was added to the residue obtained by evaporation of the solvent under reduced pressure, and the mixture was extracted with dichloromethane. The dichloromethane layer was washed with brine and dried over anhydrous sodium sulfate. The residue obtained by evaporation of the solvent under reduced pressure was purified by column chromatography on silica gel(n-hexane:ethyl acetate=3:1) to give the title compound (0.068 g, 59.2%) as an yellow oil.

¹H-NMR(CDCl₃):δ 1.36(3H, t, J=7.0 Hz), 2.50(3H, s), 4.43(2H, q, J=7.0 Hz), 7.33-7.38(1H, m), 7.44-7.50(2H, m), 7.64-7.67(2H, m), 9.91(1H, s).

### (2) Preparation of 3-[(5-ethoxy-3-methyl-1-phenylpyrazol-4-yl)methylidene]amino-2-methyl-3,4-dihydroquinazolin-4-one (Compound No. 3).

The title compound was obtained in the same manner as the Example 1(4) under the following reaction condition.

Raw materials: 3-amino-2-methyl-3,4-dihydroquinazolin-4-one (compound of Example 1(2)) and 5-ethoxy-3-methyl-1-phenylpyrazole-4-carbaldehyde.

Solvent: mixed solvent of ethanol/acetic acid.

Reaction: refluxed for 7 hours.

Yield: 19.4%.

¹H-NMR(CDCl₃):δ 1.33(3H, t, J=7.0 Hz), 2.56(3H, s), 2.66(3H, s), 4.27(2H, q, J=7.0 Hz), 7.33-7.38(1H, m), 7.43-7.50(3H, m), 7.66-7.77(4H, m), 8.23-8.31(1H, m), 8.81(1H, s).

### Example 4

### Preparation of the Compound of Compound No. 4

### (1) Preparation of 5-(ethoxycarbonyl)methoxy-3-methyl-1-phenylpyrazole-4-carbaldehyde

Sodium carbonate (2.76 g, 20 mmol) was added to a solution of 3-methyl-5-oxo-1-phenyl-4,5-dihydropyrazole-4-carbaldehyde (compound of Example 1(3); 0.404 g, 2 mmol) in acetone (20 mL), and the mixture was stirred at room temperature for 30 minutes. Bromoacetic acid ethyl ester (0.27 mL, 2.4 mmol) was added to the mixture, and the mixture was refluxed for 8 hours. After the reaction mixture was cooled to room temperature, the insoluble matter was filtered off, and the residue obtained by evaporation of the solvent under reduced pressure was purified by column chromatography on silica gel (chloroform) to give the title compound (0.214 g, 37.1%) as an yellow oil.

¹H-NMR(CDCl₃): δ 1.22 (3H, t, J=7.3 Hz), 2.45(3H, s), 4.17(2H, q, J=7.3 Hz), 5.23(2H, s), 7.34-7.49(3H, m), 7.69-7.73(2H, m), 9.78(1H, s).

### (2) Preparation of 3-{[5-(ethoxycarbonyl)methoxy-3-methyl-1-phenylpyrazol-4-yl]methylidene}amino-2-methyl-3,4-dihydroquinazolin-4-one (Compound No. 4)

The title compound was obtained in the same manner as the Example 1(4) under the following reaction condition.

Raw materials: 3-amino-2-methyl-3,4-dihydroquinazolin-4-one (compound of Example 1(2)) and 5-(ethoxycarbonyl)methoxy-3-methyl-1-phenylpyrazole-4-carbaldehyde.

Solvent: mixed solvent of xylene/acetic acid.

Reaction: refluxed for 24 hours.

Yield: 29.0%.

¹H-NMR(CDCl₃): δ 1.18(3H, t, J=7.3 Hz), 2.50(3H, s), 2.63(3H, s), 4.16(2H, q, J=7.1 Hz), 4.92(2H, s), 7.34-7.51(4H, m), 7.66-7.77(4H, m), 8.28(1H, dd, J=7.9, 1.0 Hz), 8.78(1H, s).

### Example 5

### Preparation of the Compound of Compound No. 5

Aqueous potassium hydroxide (11.2 mg/0.1 mL) was added to a solution of 3-{[5-(ethoxycarbonyl)methoxy-3-methyl-1-phenylpyrazol-4-yl]methylidene}amino-2-m ethyl-3,4-dihydroquinazolin-4-one (Compound No. 4; 30 mg, 0.067 mmol) in methanol (3 mL), and the mixture was stirred at room temperature for 15 minutes. Distilled water was added to the residue obtained by concentration of the reaction mixture under reduced pressure, and the resulting insoluble matter was filtered off. The filtrate was acidified by addition of 1N hydrochloric acid, and the separated crystal was filtered and washed with water to give the title compound (14.7 mg, 50.8%) as a white solid.

¹H-NMR(DMSO-d₆): δ 2.41(3H, s), 2.49(3H, s), 5.11(2H, s), 7.38-7.43(1H, m), 7.50-7.56(3H, m), 7.64-7.67(1H, m), 7.75-7.85(3H, m), 8.16(1H, dd, J=7.9, 1.3 Hz), 8.79(1H, s), 13.20(1H, brs).

### Example 6

### Preparation of the Compound of Compound No. 6

### (1) Preparation of 3-ethyl-1-phenyl-4,5-dihydropyrazol-5-one

Phenylhydrazine (3.79 g, 35 mmol) was added to a solution of 3-oxopentanoic acid ethyl ester (4.90 g, 34 mmol) in ethanol (35 mL), and the mixture was refluxed for 8 hours. After the reaction mixture was cooled to room temperature, the separated solid was filtered and washed with ethanol to give the title compound (6.16 g, 96.2%) as a white crystal.

¹H-NMR(CDCl₃): δ 1.25(3H, t, J=7.6 Hz), 2.52(2H, q, J=7.6 Hz), 3.42(2H, s), 7.13-7.20(1H, m), 7.35-7.41(2H, m), 7.86-7.89(2H, m).

### (2) Preparation of 3-ethyl-5-oxo-1-phenyl-4,5-dihydropyrazole-4-carbaldehyde

The title compound was obtained in the same manner as the Example 1(3) using 3-ethyl-1-phenyl-4,5-dihydropyrazol-5-one as the raw material.

Yield: 80.6%.

¹H-NMR(CDCl₃): δ 1.37(3H, t, J=7.6 Hz), 2.81(2H, q, J=7.6 Hz), 4.87(1H, br), 7.27-7.33(1H, m), 7.43-7.49(2H, m), 7.82(2H, d, J=7.6 Hz), 9.56(1H, s).

### (3) Preparation of 3-[(3-ethyl-5-hydroxy-1-phenylpyrazol-4-yl)methylidene]-amino-2-methyl-3,4-dihydroquinazolin-4-one (Compound No. 6)

The title compound was obtained in the same manner as the Example 1(4) using 3-amino-2-methyl-3,4-dihydroquinazolin-4-one (compound of Example 1(2)) and 3-ethyl-5-oxo-1-phenyl-4,5-dihydropyrazole-4-carbaldehyde as the raw materials.

Yield: 89.5%.

¹H-NMR(CD₃OD): δ 1.28(3H, t, J=7.4 Hz), 2.59-2.69(5H, m), 6.92(1H, brs), 7.08(1H, t, J=7.1 Hz), 7.24(1H, t, J=7.6 Hz), 7.34(1H, brs), 7.51(1H, t, J=7.6 Hz), 7.62-7.64(1H, m), 7.77-7.84(2H, m), 8.16-8.18(2H, m).

### Example 7

### Preparation of the Compound of Compound No. 7

### (1) Preparation of 5-oxo-1-phenyl-3-trifluoromethyl-4,5-dihydropyrazole-4-carbaldehyde

The title compound was obtained in the same manner as the Example 1(3) using 1-phenyl-3-trifluoromethyl-4,5-dihydropyrazol-5-one as the raw material.

Yield: 14.8%.

¹H-NMR(CDCl₃): δ 5.47(1H, brs), 7.37-7.52(3H, m), 7.80(2H, d, J=7.6 Hz), 9.78(1H, s).

### (2) Preparation of 3-[(5-hydroxy-1-phenyl-3-(trifluoromethyl)pyrazol-4-yl)-methylidene]amino-2-methyl-3,4-dihydroquinazolin-4-one (Compound No. 7)

The title compound was obtained in the same manner as the Example 1(4) under the following reaction condition.

Raw materials: 3-amino-2-methyl-3,4-dihydroquinazolin-4-one (compound of Example 1(2)) and 5-oxo-1-phenyl-3-trifluoromethyl-4,5-dihydropyrazole-4-carbaldehyde.

Solvent: mixed solvent of ethanol/acetic acid.

Reaction: stirred at room temperature for 2 hours.

Yield: 56.4%.

¹H-NMR(CDCl₃): δ 2.57(3H, s), 6.96-7.77(8H, m), 8.14-8.27(2H, m).

### Example 8

### Preparation of the Compound of Compound No. 8

### (1) Preparation of 3-[2-(ethoxycarbonyl)ethyl]-1-phenyl-4,5-dihydropyrazol-5-one

The title compound was obtained in the same manner as the Example 6(1) using β-ketoadipic acid diethyl ester and phenylhydrazine as the raw materials.

Yield: 92.1%.

¹H-NMR(CDCl₃): δ 1.28(3H, t, J=7.3 Hz), 2.72-2.84(4H, m), 3.46(2H, s), 4.18(2H, q, J=7.3 Hz), 7.15-7.20(1H, m), 7.35-7.41(2H, m), 7.85(2H, d, J=7.6 Hz).

### (2) Preparation of 3-[2-(ethoxycarbonyl)ethyl]-5-oxo-1-phenyl-4,5-dihydropyrazole-4-carbaldehyde

The title compound was obtained in the same manner as the Example 1(3) using 3-[2-(ethoxycarbonyl)ethyl]-1-phenyl-4,5-dihydropyrazol-5-one as the raw material.

Yield: 70.7%.

¹H-NMR(CDCl₃): δ 1.27(3H, t, J=7.3 Hz), 2.84(2H, t, J=7.3 Hz), 3.11(2H, t, J=7.3 Hz), 4.17(2H, q, J=7.3 Hz), 7.26-7.33(1H, m), 7.42-7.53(2H, m), 7.80(2H, d, J=7.8 Hz), 8.56(1H, brs), 9.59(1H, s).

### (3) Preparation of 3-({3-[2-(ethoxycarbonyl)ethyl]-5-hydroxy-1-phenylpyrazol-4-yl}methylidene)amino-2-methyl-3,4-dihydroquinazolin-4-one (Compound No. 8)

The title compound was obtained in the same manner as the Example 1(4) using 3-amino-2-methyl-3,4-dihydroquinazolin-4-one (compound of Example 1(2)) and 3-[2-(ethoxycarbonyl)ethyl]-5-oxo-1-phenyl-4,5-dihydropyrazole-4-carbaldehyde as the raw materials.

Yield: 68.5%.

¹H-NMR(CD₃OD): δ 1.22(3H, t, J=7.3 Hz), 2.60(3H, s), 2.76(2H, t, J=7.3 Hz), 2.93(2H, t, J=7.3 Hz), 4.12(2H, q, J=7.3 Hz), 7.08-7.11(1H, m), 7.23-7.35(2H, m), 7.49-7.65(2H, m), 7.80-7.85(3H, m), 8.17-8.19(2H, m).

### Example 9

### Preparation of the Compound of Compound No. 9

The title compound was obtained in the same manner as the Example 5 under the following reaction condition.

Raw material:

### 3-({3-[2-(ethoxycarbonyl)ethyl]-5-hydroxy-1-phenylpyrazol-4-yl}methylidene)amino-2-methyl-3,4-dihydroquinazolin-4-one (Compound No. 8)

Base: aqueous sodium hydroxide.

Solvent: methanol.

Reaction: 50° C., for 3 hours.

Yield: 87.7%.

¹H-NMR(CD₃OD): δ 2.51(3H, s), 2.67(2H, t, J=6.8 Hz), 2.81(2H, t, J=6.6 Hz), 7.02-7.08(1H, m), 7.23-7.29(2H, m), 7.42-7.47(1H, m), 7.53-7.56(1H, m), 7.71-7.81(3H, m), 8.02(1H, s), 8.11(1H, d, J=7.6 Hz).

### Example 10

### Preparation of the Compound of Compound No. 10

1 mol/L solution of trimethylaluminium/n-hexane (2 mL, 2 mmol) and a solution of 3-{[3-(2-carboxyethyl)-5-hydroxy-1-phenylpyrazol-4-yl]methylidene}-amino-2-methyl-3,4-dihydroquinazolin-4-one (Compound No. 9; 0.0891 g, 0.2 mmol) in benzene were added successively to a suspension of ammonium chloride (0.053 g, 1 mmol) in benzene, and the mixture was stirred at room temperature for 1 hour, then at 50° C. for 6 hours. After the reaction mixture was cooled to room temperature, it was poured into ice and water, and extracted with benzene. The benzene layer was washed with brine and dried over anhydrous sodium sulfate. The residue obtained by evaporation of the solvent under reduced pressure was purified by column chromatography on silica gel (chloroform) to give the title compound (0.0487 g, 58.5%) as a light yellow powder.

¹H-NMR (DMSO-d₆): δ 2.50-2.54(5H, m), 2.87(2H, t, J=7.8 Hz), 6.74(1H, s), 7.03(1H, t, J=6.9 Hz), 7.32(3H, m), 7.49(1H, t, J=7.4 Hz), 7.61-7.65(1H, m), 7.80(1H, t, J=7.1 Hz), 8.04-8.13(3H, m), 3.12(1H, s).

### Example 11

### Preparation of the Compound of Compound No. 11

### (1) Preparation of 3-[3-(ethoxycarbonyl)propyl]-1-phenyl-4,5-dihydropyrazol-5-one

The title compound was obtained in the same manner as the Example 6(1) using 3-oxopimelic acid diethyl ester and phenylhydrazine as the raw materials.

Yield: 76.8%.

¹H-NMR(CDCl₃): δ 1.25(3H, t, J=7.3 Hz), 1.96-2.07(2H, m), 2.45(2H, t, J=7.3 Hz), 2.55(2H, t, J=7.3 Hz), 3.44(2H, s), 4.14(2H, q, J=7.3 Hz), 7.15-7.20(1H, m), 7.36-7.42(2H, m), 7.84-7.88(2H, m).

### (2) Preparation of 3-[3-(ethoxycarbonyl)propyl]-5-oxo-1-phenyl-4,5-dihydropyrazole-4-carbaldehyde

The title compound was obtained in the same manner as the Example 1(3) using 3-[3-(ethoxycarbonyl)propyl]-1-phenyl-4,5-dihydropyrazol-5-one as the raw material.

Yield: 100%.

¹H-NMR(CDCl₃): δ 1.27(3H, t, J=7.1 Hz), 2.08-2.15(2H, m), 2.43-2.51(2H, m), 2.81-2.89(2H, m), 4.15(2H, q, J=7.1 Hz), 5.17(1H, brs), 7.27-7.34(1H, m), 7.43-7.50(2H, m), 7.78-7.82(2H, m), 9.57(1H, s).

### (3) Preparation of 3-({3-[3-(ethoxycarbonyl)propyl]-5-hydroxy-1-phenylpyrazol-4-yl}methylidene)amino-2-methyl-3,4-dihydroquinazolin-4-one (Compound No. 11)

The title compound was obtained in the same manner as the Example 1(4) using 3-amino-2-methyl-3,4-dihydroquinazolin-4-one (compound of Example 1(2)) and 3-[3-(ethoxycarbonyl)propyl]-5-oxo-1-phenyl-4,5-dihydropyrazole-4-carbaldehyde as the raw materials.

Yield: 64.1%.

¹H-NMR(CD₃OD): δ 1.18(3H, t, J=7.1 Hz), 1.96(2H, t, J=7.0 Hz), 2.41(2H, t, J=7.0 Hz), 2.52-2.69(5H, m), 4.07(2H, q, J=7.1 Hz), 6.83-7.09(3H, m), 7.34-7.49(2H, m), 7.58-7.81(3H, m), 8.11-8.14(1H, m), 8.23-8.31(1H, m).

### Example 12

### Preparation of the Compound of Compound No. 12

The title compound was obtained in the same manner as the Example 5 under the following reaction condition.

Raw material: 3-({3-[3-(ethoxycarbonyl)propyl]-5-hydroxy-1-phenylpyrazol-4-yl-}methylidene)amino-2-methyl-3,4-dihydroquinazolin-4-one (Compound No. 11).

Base: aqueous sodium hydroxide.

Solvent: methanol.

Reaction: 50° C., for 3 hours.

Yield: 58.8%.

¹H-NMR(CD₃OD): δ 2.02(2H, t, J=7.4 Hz), 2.44(2H, t, J=7.1 Hz), 2.62(3H, s), 2.69(2H, t, J=7.5 Hz), 7.19(1H, t, J=7.4 Hz), 7.38(2H, t, J=8.0 Hz), 7.56(1H, t, J=7.5 Hz), 7.67(1H, d, J=8.2 Hz), 7.83-7.87(3H, m), 8.12(1H, s), 8.21-8.24(1H, m).

### Example 13

### Preparation of the Compound of Compound No. 13

The title compound was obtained in the same manner as the Example 10 using 3-{[3-(3-carboxypropyl)-5-hydroxy-1-phenylpyrazol-4-yl]methylidene}amino-2-methyl-3,4-dihydroquinazolin-4-one (Compound No. 12) as the raw material.

Yield: 51.7%.

¹H-NMR (DMSO-d₆): δ 1.77-1.80(2H, m), 2.11(2H, t, J=7.1 Hz), 2.50-2.54(5H, m), 6.71-6.85(4H, m), 7.23-7.48(5H, m), 7.68(1H, t, J=7.4 Hz), 8.11(1H, d, J=7.6 Hz), 8.53(1H, s).

### Example 14

### Preparation of the Compound of Compound No. 14

### (1) Preparation of 1,3-dimethyl-5-oxo-4,5-dihydropyrazole-4-carbaldehyde

The title compound was obtained in the same manner as the Example 1(3) using 1,3-dimethyl-4,5-dihydropyrazol-5-one as the raw material.

Yield: 6.2%.

¹H-NMR (DMSO-d₆): δ 2.22(3H, s), 3.43(3H, s), 9.64(1H, s).

### (2) Preparation of 3-[(1,3-dimethyl-5-hydroxypyrazol-4-yl)methylidene]amino-2-methyl-3,4-dihydroquinazolin-4-one (Compound No. 14)

The title compound was obtained in the same manner as the Example 1(4) using 3-amino-2-methyl-3,4-dihydroquinazolin-4-one (compound of Example 1(2)) and 1,3-dimethyl-5-oxo-4,5-dihydropyrazole-4-carbaldehyde as the raw materials.

Yield: 67.6%.

¹H-NMR (DMSO-d₆): δ 2.12(3H, s), 2.51(3H, s), 3.31(3H, s), 7.54(1H, t, J=7.4 Hz), 7.64-7.67(1H, m), 7.82-7.88(1H, m), 8.11-8.16(2H, m).

### Example 15

### Preparation of the Compound of Compound No. 15

### (1) Preparation of 1-(2-chlorophenyl)-3-methyl-5-oxo-4,5-dihydropyrazole-4-carbaldehyde

The title compound was obtained in the same manner as the Example 1(3) using 1-(2-chlorophenyl)-3-methyl-4,5-dihydropyrazol-5-one as the raw material.

Yield: 54.2%.

¹H-NMR(CDCl₃): δ 2.44(3H, s), 7.38-7.47(3H, m), 7.53-7.57(1H, m), 8.36(1H, brs), 9.57(1H, s).

### (2) Preparation of 3-{[1-(2-chlorophenyl)-3-methyl-5-hydroxypyrazol-4-yl]-methylidene}amino-2-methyl-3,4-dihydroquinazolin-4-one (Compound No. 15)

The title compound was obtained in the same manner as the Example 1(4) using 3-amino-2-methyl-3,4-dihydroquinazolin-4-one (compound of Example 1(2)) and 1-(2-chlorophenyl)-3-methyl-5-oxo-4,5-dihydropyrazole-4-carbaldehyde as the raw materials.

Yield: 71.8%.

¹H-NMR (DMSO-d₆): δ 2.15(3H, s), 2.49(3H, s), 7.43-7.69(6H, m), 7.85-7.90(1H, m), 8.13-8.18(2H, m).

### Example 16

### Preparation of the Compound of Compound No. 16

### (1) Preparation of 1-(3-chlorophenyl)-3-methyl-4,5-dihydropyrazol-5-one

The title compound was obtained in the same manner as the Example 6(1) using acetoacetic acid ethyl ester and (3-chlorophenyl)hydrazine as the raw materials.

Yield: 35.6%.

¹H-NMR(CDCl₃): δ 2.20(3H, s), 3.44(2H, s), 7.12-7.16(1H, m), 7.28-7.34(1H, m), 7.81-7.85(1H, m), 7.92-7.94(1H, m).

### (2) Preparation of 1-(3-chlorophenyl)-3-methyl-5-oxo-4,5-dihydropyrazole-4-carbaldehyde

The title compound was obtained in the same manner as the Example 1(3) using 1-(3-chlorophenyl)-3-methyl-4,5-dihydropyrazol-5-one as the raw material.

Yield: 83.9%.

¹H-NMR(CDCl₃): δ 2.42(3H, s), 7.25-7.28(1H, m), 7.46-7.47(1H, m), 7.74-7.77(1H, m), 7.88-7.90(1H, m), 9.01(1H, brs), 9.40(1H, s).

### (3) Preparation of 3-{[1-(3-chlorophenyl)-3-methyl-5-hydroxypyrazol-4-yl]-methylidene}amino-2-methyl-3,4-dihydroquinazolin-4-one (Compound No. 16)

The title compound was obtained in the same manner as the Example 1(4) using 3-amino-2-methyl-3,4-dihydroquinazolin-4-one (compound of Example 1(2)) and 1-(3-chlorophenyl)-3-methyl-5-oxo-4,5-dihydropyrazole-4-carbaldehyde as the raw materials.

Yield: 52.9%.

¹H-NMR (DMSO-d₆): δ 2.19(3H, s), 2.50(3H, s), 7.19(1H, d, J=8.3 Hz), 7.44(1H, t, J=8.3 Hz), 7.56(1H, t, J=7.9 Hz), 7.68(1H, d, J=7.9 Hz), 7.85-7.90(1H, m), 7.96-7.99(1H, m), 8.10-8.16(2H, m), 8.21(1H, s).

### Example 17

### Preparation of the Compound of Compound No. 17

### (1) Preparation of 1-(4-chlorophenyl)-3-methyl-4,5-dihydropyrazol-5-one

The title compound was obtained in the same manner as the Example 6(1) using acetoacetic acid ethyl ester and (4-chlorophenyl)hydrazine as the raw materials.

Yield: 45.4%.

¹H-NMR(CDCl₃): δ 2.20(3H, s), 3.44(2H, s), 7.33-7.37(2H, m), 7.82-7.86(2H, m).

### (2) Preparation of 1-(4-chlorophenyl)-3-methyl-5-oxo-4,5-dihydropyrazole-4-carbaldehyde

The title compound was obtained in the same manner as the Example 1(3) using 1-(4-chlorophenyl)-3-methyl-4,5-dihydropyrazol-5-one as the raw material.

Yield: 90.4%.

¹H-NMR(CDCl₃): δ 2.41(3H, s), 4.17(1H, s), 7.40-7.42(2H, m), 7.73-7.81(2H, m), 9.44(1H, s).

### (3) Preparation of 3-{[1-(4-chlorophenyl)-3-methyl-5-hydroxypyrazol-4-yl]methylidene}amino-2-methyl-3,4-dihydroquinazolin-4-one (Compound No. 17)

The title compound was obtained in the same manner as the Example 1(4) using 3-amino-2-methyl-3,4-dihydroquinazolin-4-one (compound of Example 1(2)) and 1-(4-chlorophenyl)-3-methyl-5-oxo-4,5-dihydropyrazole-4-carbaldehyde as the raw materials.

Yield: 67.0%.

¹H-NMR(CDCl₃): δ 2.23(3H, s), 2.62(3H, s), 7.19-7.23(2H, m), 7.44-7.52(2H, m), 7.60(1H, d, J=8.3 Hz), 7.75-7.81(3H, m), 8.16-8.19(1H, m).

### Example 18

### Preparation of the Compound of Compound No. 18

### (1) Preparation of 3-methyl-1-(4-methylphenyl)-5-oxo-4,5-dihydropyrazole-4-carbaldehyde

The title compound was obtained in the same manner as the Example 1(3) using 3-methyl-1-(4-methylphenyl)-4,5-dihydropyrazol-5-one as the raw material.

Yield: 45.8%.

¹H-NMR(CDCl₃): δ 2.38(3H, s), 2.42(3H, s), 6.66(1H, brs), 7.26(2H, d, J=8.6 Hz), 7.66(2H, d, J=8.6 HZ), 9.55(1H, s).

### (2) Preparation of 3-{[3-methyl-1-(4-methylphenyl)-5-hydroxypyrazol-4-yl]-methylidene}amino-2-methyl-3,4-dihydroquinazolin-4-one (Compound No. 18).

The title compound was obtained in the same manner as the Example 1(4) under the following reaction condition.

Raw materials: 3-amino-2-methyl-3,4-dihydroquinazolin-4-one (compound of Example 1(2)) and 3-methyl-1-(4-methylphenyl)-5-oxo-4,5-dihydropyrazole-4-carbaldehyde.

Solvent: mixed solvent of ethanol/acetic acid.

Reaction: 40-50° C., for 2 hours.

Yield: 63.3%.

¹H-NMR(DMSO-d₆): δ 2.20(3H, s), 2.29(3H, s), 7.18(2H, d, J=8.6 Hz), 7.53(1H, t, J=7.6 Hz), 7.66(1H, d, J=8.3 Hz), 7.82-7.89(3H, m), 8.12-8.15(2H, m).

### Example 19

### Preparation of the Compound of Compound No. 19

### (1) Preparation of 3-methyl-1-(4-methoxyphenyl)-4,5-dihydropyrazol-5-one

The title compound was obtained in the same manner as the Example 6(1) using acetoacetic acid ethyl ester and (4-methoxyphenyl)hydrazine as the raw materials.

Yield: 29.0%.

¹H-NMR(CDCl₃): δ 2.19(3H, s), 3.40(2H, s), 3.81(3H, s), 6.89-6.94(2H, m), 7.70-7.76(2H, m).

### (2) Preparation of 3-methyl-1-(4-methoxyphenyl)-5-oxo-4,5-dihydropyrazole-4-carbaldehyde

The title compound was obtained in the same manner as the Example 1(3) using 3-methyl-1-(4-methoxyphenyl)-4,5-dihydropyrazol-5-one as the raw material.

Yield: 54.5%.

¹H-NMR(CDCl₃): δ 2.42(3H, s), 3.84(3H, s), 6.94-6.99(2H, m), 7.64-7.69(2H, m), 9.35(1H, s), 9.56(1H, s).

### (3) Preparation of 3-{[3-methyl-1-(4-methoxyphenyl)-5-hydroxypyrazol-4-yl]-methylidene}amino-2-methyl-3,4-dihydroquinazolin-4-one (Compound No. 19)

The title compound was obtained in the same manner as the Example 1(4) using 3-amino-2-methyl-3,4-dihydroquinazolin-4-one (compound of Example 1(2)) and 3-methyl-1-(4-methoxyphenyl)-5-oxo-4,5-dihydropyrazole-4-carbaldehyde as the raw materials.

Yield: 77.0%.

¹H-NMR(DMSO-d₆): δ 2.16(3H, s), 2.49(3H, s), 3.43(3H, s), 6.99(2H, d, J=8.9 Hz), 7.56(1H, t, J=7.4 Hz), 7.68(1H, d, J=7.9 Hz), 7.83-7.90(3H, m), 8.13-8.17(2H, m).

### Example 20

### Preparation of the Compound of Compound No. 20.

### (1) Preparation of 3-methyl-1-(4-nitrophenyl)-5-oxo-4,5-dihydropyrazole-4-carbaldehyde

The title compound was obtained in the same manner as the Example 1(3) using 3-methyl-1-(4-nitrophenyl)-4,5-dihydropyrazol-5-one as the raw material.

Yield: 72.0%. ¹H-NMR(CDCl₃): δ 2.56(3H, s), 7.05(1H, brs), 7.83-7.87(2H, m), 8.39-8.42(2H, m), 10.01(1H, s).

### (2) Preparation of 3-{[3-methyl-1-(4-nitrophenyl)-5-hydroxypyrazol-4-yl]-methylidene}amino-2-methyl-3,4-dihydroquinazolin-4-one (Compound No. 20)

The title compound was obtained in the same manner as the Example 1(4) using 3-amino-2-methyl-3,4-dihydroquinazolin-4-one (compound of Example 1(2)) and 3-methyl-1-(4-nitrophenyl)-5-oxo-4,5-dihydropyrazole-4-carbaldehyde as the raw materials.

Yield: 73.3%.

¹H-NMR(DMSO-d₆): δ 2.50(3H, s), 2.51(3H, s), 7.47(1H, t, J=7.3 Hz), 7.61(1H, d, J=7.9 Hz), 7.77(1H, t, J=7.9 Hz), 8.05(1H, s), 8.11(1H, d, J=7.7 Hz), 8.19(2H, d, J=8.6 Hz), 8.35(2H, d, J=8.6 Hz).

### Example 21

### Preparation of the Compound of Compound No. 21

The title compound was obtained in the same manner as the Example 1(4) under the following reaction condition.

Raw materials: 3-amino-2-ethyl-3,4-dihydroquinazolin-4-one and 3-methyl-5-oxo-1-phenyl-4,5-dihydropyrazole-4-carbaldehyde (compound of Example 1(3)).

Solvent: mixed solvent of ethanol/acetic acid.

Reaction: 50-60° C., for 2 hours.

Yield: 87.1%.

¹H-NMR(CDCl₃): δ 1.39(3H, t, J=7.3 Hz), 2.25(3H, s), 2.92(2H, q, J=7.3 Hz), 5.09(1H, br), 7.11-7.16(3H, m), 7.32-7.38(2H, m), 7.46-7.52(2H, m), 7.67-7.82(2H, m), 7.87-7.90(2H, m), 8.22(1H, dd, J=8.0, 1.4 Hz).

### Example 22

### Preparation of the Compound of Compound No. 22

### (1) 2-(Isobutyrylamino)benzoic acid methyl ester

The title compound was obtained in the same manner as the Example 1(1) using anthranilic acid methyl ester and isobutyryl chloride as the raw materials.

Yield: 100%.

¹H-NMR(CDCl₃): δ 1.30(6H, d, J=7.7 Hz), 2.58-2.68(1H, m), 3.93(3H, s), 7.04-7.09(1H, m), 7.51-7.57(1H, m), 8.01-8.05(1H, m), 8.74-8.77(1H, m), 11.1(1H, brs).

### (2) 3-Amino-2-isopropyl-3,4-dihydroquinazolin-4-one

The title compound was obtained in the same manner as the Example 1(2) using 2-(isobutyrylamino)benzoic acid methyl ester and hydrazine monohydrate as the raw materials.

Yield: 56.5%.

¹H-NMR(CDCl₃): δ 1.37(6H, d, J=6.9 Hz), 3.72-3.81(1H, m), 4.83(2H, s), 7.41-7.47(1H, m), 7.68-7.76(2H, m), 8.29(1H, d, J=8.0 Hz).

### (3) Preparation of 3-[(5-hydroxy-3-methyl-1-phenylpyrazol-4-yl)methylidene]-amino-2-isopropyl-3,4-dihydroquinazolin-4-one (Compound No. 22).

The title compound was obtained in the same manner as the Example 1(4) using 3-amino-2-isopropyl-3,4-dihydroquinazolin-4-one and 3-methyl-5-oxo-1-phenyl-4,5-dihydropyrazole-4-carbaldehyde (compound of Example 1(3)) as the raw materials.

Yield: 54.2%.

¹H-NMR(CDCl₃):δ 1.37(6H, d, J=6.6 Hz), 2.25(3H, s), 3.33-3.42(1H, m), 5.44(1H, brs), 7.11-7.16(1H, m), 7.33-7.38(2H, m), 7.46-7.51(2H, m), 7.68-7.81(2H, m), 7.89-7.92(2H, m), 8.21-8.24(1H, m).

### Example 23

### Preparation of the Compound of Compound No. 23

### (1) 2-(Valerylamino)benzoic acid methyl ester

The title compound was obtained in the same manner as the Example 1(1) using anthranilic acid methyl ester and valeryl chloride as the raw materials.

Yield: 100%.

¹H-NMR(CDCl₃): δ 0.97(3H, t, J=7.3 Hz), 1.39-1.46(2H, m), 1.70-1.77(2H, m), 2.45(2H, t, J=7.6 Hz), 3.93(3H, s), 7.04-7.10(1H, m), 7.51-7.57(1H, m), 8.01-8.04(1H, m), 8.73-8.76(1H, m), 11.06(1H, brs).

### (2) 3-Amino-2-butyl-3,4-dihydroquinazolin-4-one

The title compound was obtained in the same manner as the Example 1(2) using 2-(valerylamino)benzoic acid methyl ester and hydrazine monohydrate as the raw materials.

Yield: 70.6%.

¹H-NMR(CDCl₃): δ 0.99(3H, t, J=7.3 Hz), 1.44-1.56(2H, m), 1.77-1.87(2H, m), 3.03(2H, t, J=8.0 Hz), 4.86(2H, s), 7.41-7.47(1H, m), 7.65-7.76(2H, m), 8.22-8.25(1H, m).

### (3) Preparation of 2-butyl-3-[(5-hydroxy-3-methyl-1-phenylpyrazol-4-yl)-methylidene]amino-3,4-dihydroquinazolin-4-one (Compound No. 23).

The title compound was obtained in the same manner as the Example 1(4) using 3-amino-2-butyl-3,4-dihydroquinazolin-4-one and 3-methyl-5-oxo-1-phenyl-4,5-dihydropyrazole-4-carbaldehyde (compound of Example 1(3)) as the raw materials.

Yield: 56.1%.

¹H-NMR(DMSO-d₆): δ 0.96(3H, t, J=7.4 Hz), 1.32-1.45(2H, m), 1.68-1.78(2H, m), 2.25(3H, s), 2.81(2H, t, J=7.7 Hz), 3.64(1H, brs), 7.04(1H, t, J=7.0 Hz), 7.33(2H, t, J=7.7 Hz), 7.50(1H, t, J=7.6 Hz), 7.65(1H, d, J=8.2 Hz), 7.77-7.82(1H, m), 8.02-8.14(4H, m).

### Example 24

### Preparation of the Compound of Compound No. 24

### (1) 2-Phenyl-4H-3,1-benzoxazin-4-one

Benzoyl chloride (2.32 mL, 20 mmol) was added to a solution of anthranilic acid (1.37 g, 10 mmol) in pyridine (30 mL) under ice cooling, and the mixture was stirred at 60° C. for 7 hours. After the reaction mixture was cooled to room temperature, it was poured into ice and water. After the separated crystal was filtered and washed with water, it was recrystallized from methanol to give the title compound (1.95 g, 87.5%) as a white crystal.

¹H-NMR(CDCl₃): δ 7.49-7.60(4H, m), 7.61-7.87(2H, m), 8.24-8.34(3H, m).

### (2) 3-Amino-2-phenyl-3,4-dihydroquinazolin-4-one

Hydrazine monohydrate (0.44 mL, 9 mmol) was added to a solution of 2-phenyl-4H-3,1-benzoxazin-4-one (1.00 g, 4.48 mmol) in ethanol (20 mL), and the mixture was refluxed for 12 hours. After the reaction mixture was cooled to room temperature, the solvent was evaporated under reduced pressure, xylene was added, and the mixture was refluxed for 12 hours. After the reaction mixture was cooled to room temperature, the solid obtained by evaporation of the solvent under reduced pressure was washed with methanol under suspension to give the title compound (0.77 g, 72.6%) as a white crystal.

¹H-NMR(CDCl₃): δ 5.02(2H, S), 7.48-7.57(4H, m), 7.78-7.81(4H, m), 8.31-8.34(1H, m).

### (3) Preparation of 3-[(5-hydroxy-3-methyl-1-phenylpyrazol-4-yl)methylidene]-amino-2-phenyl-3,4-dihydroquinazolin-4-one (Compound No. 24)

The title compound was obtained in the same manner as the Example 1(4) using 3-amino-2-phenyl-3,4-dihydroquinazolin-4-one and 3-methyl-5-oxo-1-phenyl-4,5-dihydropyrazole-4-carbaldehyde (compound of Example 1(3)) as the raw materials.

Yield: 77.8%.

¹H-NMR(CDCl₃): δ 2.17(3H, s), 7.10-7.13(1H, m), 7.29-7.34(2H, m), 7.47-7.58(5H, m), 7.68-7.72(2H, m), 7.80-7.83(4H, m), 8.30(1H, d, J=8.2 Hz).

### Example 25

### Preparation of the Compound of Compound No. 25

### (1) 2-tert-Butyl-4H-3,1-benzoxazin-4-one

The title compound was obtained in the same manner as the Example 24(1) using anthranilic acid and pivaloyl chloride as the raw materials.

Yield: 100%.

¹H-NMR(CDCl₃): δ 1.40(9H, s), 7.46-7.52(1H, m), 7.57-7.60(1H, m), 7.75-7.81(1H, m), 8.17-8.20(1H, m).

### (2) 3-Amino-2-tert-butyl-3,4-dihydroquinazolin-4-one

The title compound was obtained in the same manner as the Example 24(2) under the following reaction condition.

Raw materials: 2-tert-butyl-4H-3,1-benzoxazin-4-one and hydrazine monohydrate.

Solvent: xylene.

Reaction: refluxed for 20 hours.

Yield: 75.1%.

¹H-NMR(CDCl₃): δ 1.58(9H, s), 4.74(2H, s), 7.41-7.47(1H, m), 7.66-7.76(2H, m), 8.23(1H, d, J=8.3 Hz).

### (3) Preparation of 2-tert-butyl-3-[(5-hydroxy-3-methyl-1-phenylpyrazol-4-yl)methylidene]amino-3,4-dihydroquinazolin-4-one (Compound No. 25)

The title compound was obtained in the same manner as the Example 1(4) using 3-amino-2-tert-butyl-3,4-dihydroquinazolin-4-one and 3-methyl-5-oxo-1-phenyl-4,5-dihydropyrazole-4-carbaldehyde (compound of Example 1(3)) as the raw materials.

Yield: 39.8%.

¹H-NMR(CDCl₃): δ 1.55(9H, s), 2.26(3H, s), 3.30(1H, brs), 7.18(1H, t, J=7.4 Hz), 7.38-7.54(4H, m), 7.71-7.83(2H, m), 7.96(2H, d, J=7.6 Hz), 8.22-8.25(1H, m).

### Example 26

### Preparation of the Compound of Compound No. 26

### (1) 2-[(Acetoxyacetyl)amino]benzoic acid methyl ester

The title compound was obtained in the same manner as the Example 1(1) using anthranilic acid methyl ester and acetoxyacetyl chloride as the raw materials.

Yield: 97.3%.

¹H-NMR(CDCl₃): δ 2.33(3H, s), 3.93(3H, s), 4.74(2H, s), 7.10-7.16(1H, m), 7.54-7.59(1H, m), 8.03-8.06(1H, m), 8.72-8.76(1H, m), 11.70(1H, br).

### (2) 3-Amino-2-hydroxymethyl-3,4-dihydroquinazolin-4-one

The title compound was obtained in the same manner as the Example 1(2) using 2-[(acetoxyacetyl)amino]benzoic acid methyl ester and hydrazine monohydrate as the raw materials.

Yield: 88.5%.

¹H-NMR(DMSO-d₆): δ 4.69(2H, d, J=5.6 Hz), 5.14(1H, t, J=5.6 Hz), 5.69(2H, s), 7.50-7.56(1H, m), 7.70(1H, d, J=7.9 Hz), 7.80-7.86(1H, m), 8.15(1H, dd, J=8.1, 1.2 Hz).

### (3) Preparation of 3-[(5-hydroxy-3-methyl-1-phenylpyrazol-4-yl)methylidene]-amino-2-hydroxymethyl-3,4-dihydroquinazolin-4-one (Compound No. 26)

The title compound was obtained in the same manner as the Example 1(4) using 3-amino-2-hydroxymethyl-3,4-dihydroquinazolin-4-one and 3-methyl-5-oxo-1-phenyl-4,5-dihydropyrazole-4-carbaldehyde (compound of Example 1(3)) as the raw materials.

Yield: 51.9%.

¹H-NMR(CDCl₃): δ 2.25(3H, s), 4.73(2H, br), 4.84(2H, s), 7.15(1H, t, J=7.4 Hz), 7.35(1H, t, J=7.9 Hz), 7.47-7.57(1H, m), 7.65(1H, s), 7.69-7.85(4H, m), 8.26(1H, d, J=7.9 Hz).

### Example 27

### Preparation of the Compound of Compound No. 27

The title compound was obtained in the same manner as the Example 1(4) under the following reaction condition.

Raw materials: 3-amino-2-hydroxymethyl-3,4-dihydroquinazolin-4-one (compound of Example 26(2)) and 5-(ethoxycarbonyl)methoxy-3-methyl-1-phenylpyrazole-4-carbaldehyde (compound of Example 4(1)).

Solvent: mixed solvent of ethanol/acetic acid.

Reaction: refluxed for 12 hours.

Yield: 54.1%.

¹H-NMR(CDCl₃): δ 1.20(3H, t, J=7.1 Hz), 2.50(3H, s), 4.13-4.21(3H, m), 4.75(2H, d, J=4.6 Hz), 4.83(2H, s), 7.35-7.54(4H, m), 7.72-7.82(4H, m), 8.30-8.34(1H, m), 9.07(1H, s).

### Example 28

### Preparation of the Compound of Compound No. 28

The title compound was obtained in the same manner as the Example 1(4) under the following reaction condition.

Raw materials: 3-amino-2-hydroxymethyl-3,4-dihydroquinazolin-4-one (compound of Example 26(2)) and

5-oxo-1-phenyl-3-trifluoromethyl-4,5-dihydropyrazole-4-carbaldehyde (compound of Example 7(1)).

Solvent: mixed solvent of ethanol/acetic acid.

Reaction: refluxed for 4.5 hours.

Yield: 21.3%.

MS (E1) m/z: 429(M+), 255, 253, 176, 174, 77.

### Example 29

### Preparation of the Compound of Compound No. 29

### (1) 2-[(methoxyacetyl)amino]benzoic acid methyl ester

The title compound was obtained in the same manner as the Example 1(1) using anthranilic acid methyl ester and methoxyacetyl chloride as the raw materials.

Yield: 97.2%.

¹H-NMR(CDCl₃): δ 3.57(3H, s), 3.95(3H, s), 4.17(2H, s), 7.09-7.15(1H, m), 7.52-7.59(1H, m), 8.05(1H, dd, J=7.9, 1.7 Hz), 8.81(1H, dd, J=8.6, 1.0 Hz), 11.73(1H, br).

### (2) 3-Amino-2-methoxymethyl-3,4-dihydroquinazolin-4-one

The title compound was obtained in the same manner as the Example 1(2) under the following reaction condition.

Raw materials: 2-[(methoxyacetyl)amino]benzoic acid methyl ester and hydrazine monohydrate.

Solvent: ethanol.

Reaction: refluxed for 15 hours.

Yield: 58.5%.

¹H-NMR(DMSO-d₆): δ 3.44(3H, s), 4.67(2H, s), 5.66(2H, s), 7.51-7.57(1H, m), 7.70(1H, d, J=8.2 Hz), 7.79-7.86(1H, m), 8.14(1H, dd, J=8.2, 1.3 Hz).

### (3) Preparation of 3-[(5-hydroxy-3-methyl-1-phenylpyrazol-4-yl)methylidene]-amino-2-methoxymethyl-3,4-dihydroquinazolin-4-one (Compound No. 29)

The title compound was obtained in the same manner as the Example 1(4) using 3-amino-2-methoxymethyl-3,4-dihydroquinazolin-4-one and 3-methyl-5-oxo-1-phenyl-4,5-dihydropyrazole-4-carbaldehyde (compound of Example 1(3)) as the raw materials.

Yield: 73.3%.

¹H-NMR(DMSO-d₆): δ 2.20(3H, s), 2.49(3H, s), 3.40(3H, s), 4.53(2H, s), 7.12(1H, t, J=7.3 Hz), 7.36-7.42(2H, m), 7.61(1H, t, J=7.3 Hz), 7.74-7.77(1H, m), 7.87-7.97(3H, m), 8.00-8.20(2H, m).

### Example 30

### Preparation of the Compound of Compound No. 30

### (1) 2-(4-Methoxyphenyl)-4H-3,1-benzoxazin-4-one

The title compound was obtained in the same manner as the Example 24(1) using anthranilic acid and 4-methoxybenzoyl chloride as the raw materials.

Yield: 85.2%.

¹H-NMR(CDCl₃): δ 3.90(3H, s), 7.00(2H, d, J=8.9 Hz), 7.45-7.50(1H, m), 7.65(1H, d, J=7.6 Hz), 7.77-7.83(1H, m), 8.20-8.28(1H, m), 8.26(2H, d, J=8.7 Hz).

### (2) 3-Amino-2-(4-methoxyphenyl)-3,4-dihydroquinazolin-4-one

The title compound was obtained in the same manner as the Example 24(2) under the following reaction condition.

Raw materials: 2-(4-methoxyphenyl)-4H-3,1-benzoxazin-4-one and hydrazine monohydrate.

Solvent: xylene.

Reaction: refluxed for 32 hours.

Yield: 90.3%.

¹H-NMR(CDCl₃): δ 3.88(3H, s), 5.05(2H, s), 6.98-7.04(2H, m), 7.46-7.52(1H, m), 7.75-7.85(4H, m), 8.28-8.31(1H, m).

### (3) Preparation of 3-[(5-hydroxy-3-methyl-1-phenylpyrazol-4-yl)methylidene]-amino-2-(4-methoxyphenyl)-3,4-dihydroquinazolin-4-one (Compound No. 30)

The title compound was obtained in the same manner as the Example 1(4) using 3-amino-2-(4-methoxyphenyl)-3,4-dihydroquinazolin-4-one and 3-methyl-5-oxo-1-phenyl-4,5-dihydropyrazole-4-carbaldehyde (compound of Example 1(3)) as the raw materials.

Yield: 59.3%.

¹H-NMR(CDCl₃): δ 2.10(3H, s), 3.79(3H, s), 7.01-7.14(3H, m), 7.38(2H, t, J=7.9 Hz), 7.62(1H, t, J=7.6 Hz), 7.74-7.79(3H, m), 7.89-7.95(3H, m), 8.18-8.22(2H, m).

### Example 31

### Preparation of the Compound of Compound No. 31

### (1) 2-[(3-Methoxybenzoyl)amino]benzic acid methyl ester

The title compound was obtained in the same manner as the Example 1(1) using anthranilic acid methyl ester and 3-methoxybenzoyl chloride as the raw materials.

Yield: 82.2%.

¹H-NMR(CDCl₃): δ 3.90(3H, s), 4.08(3H, s), 6.98-7.13(3H, m), 7.45-7.60(2H, m), 8.03(1H, dd, J=7.9, 1.7 Hz), 7.19(1H, dd, J=7.8, 1.7 Hz), 12.01(1H, br).

### (2) 3-Amino-2-(3-methoxyphenyl)-3,4-dihydroquinazolin-4-one

The title compound was obtained in the same manner as the Example 1(2) under the following reaction condition.

Raw materials: 2-[(3-methoxybenzoyl)amino]benzic acid methyl ester and hydrazine monohydrate.

Solvent: toluene.

Reaction: refluxed for 17 hours.

Yield: 94.7%.

¹H-NMR(CDCl₃): δ 3.87(3H, s), 5.04(2H, s), 7.06(1H, ddd, J=8.3, 2.4, 1.3 Hz), 7.30-7.55(4H, m), 7.77-7.80(2H, m), 8.29-8.33(1H, m).

### (3) Preparation of 3-[(5-hydroxy-3-methyl-1-phenylpyrazol-4-yl)methylidene]-amino-2-(3-methoxyphenyl)-3,4-dihydroquinazolin-4-one (Compound No. 31)

The title compound was obtained in the same manner as the Example 1(4) using 3-amino-2-(3-methoxyphenyl)-3,4-dihydroquinazolin-4-one and 3-methyl-5-oxo-1-phenyl-4,5-dihydropyrazole-4-carbaldehyde (compound of Example 1(3)) as the raw materials.

Yield: 62.0%.

¹H-NMR(CDCl₃): δ 2.17(3H, s), 3.85(3H, s), 6.85(1H, br), 7.00-7.13(2H, m), 7.22-7.59(7H, m), 7.78-7.87(4H, m), 8.30(1H, d, J=7.9 Hz).

### Example 32

### Preparation of the Compound of Compound No. 32

### (1) 2-[(3-Methoxybenzoyl)amino]benzoic acid methyl ester

The title compound was obtained in the same manner as the Example 1(1) using anthranilic acid methyl ester and 3-methoxybenzoyl chloride as the raw materials.

Yield: 100%.

¹H-NMR(CDCl₃): δ 3.92(3H, s), 4.08(3H, s), 6.98-7.13(3H, m), 7.45-7.60(2H, m), 8.03(1H, dd, J=7.9, 1.7 Hz), 8.19(1H, dd, J=7.8, 1.7 Hz), 8.91(1H, m), 12.15(1H, br).

### (2) 3-Amino-2-(2-methoxyphenyl)-3,4-dihydroquinazolin-4-one

The title compound was obtained in the same manner as the Example 1(2) under the following reaction condition. Raw materials: 2-[(2-methoxybenzoyl)amino]benzoic acid methyl ester and hydrazine monohydrate.

Solvent: toluene.

Reaction: refluxed for 24 hours.

Yield: 18.4%.

¹H-NMR(CDCl₃): δ 3.87(3H, s), 5.34(2H, s), 7.03(1H, d, J=8.3 Hz), 7.15(1H, td, J=7.6, 1.0 Hz), 7.48-7.56(3H, m), 7.77-7.80(2H, m), 8.33-8.36(1H, m).

### (3) Preparation of 3-[(5-hydroxy-3-methyl-1-phenylpyrazol-4-yl)methylidene]-amino-2-(2-methoxyphenyl)-3,4-dihydroquinazolin-4-one (Compound No. 32)

The title compound was obtained in the same manner as the Example 1(4) using 3-amino-2-(3-methoxyphenyl)-3,4-dihydroquinazolin-4-one and 3-methyl-5-oxo-1-phenyl-4,5-dihydropyrazole-4-carbaldehyde (compound of Example 1(3)) as the raw materials.

Yield: 74.5%.

¹H-NMR(CDCl₃): δ 2.14(3H, s), 4.01(3H, s), 6.98-7.17(3H, m), 7.34-7.40(3H, m), 7.46-7.61(4H, m), 7.80-7.93(4H, m), 8.34-8.37(1H, m).

### Example 33

### Preparation of the Compound of Compound No. 33

### (1) 2,5-Dimethyl-4H-3,1-benzoxazin-4-one

The title compound was obtained in the same manner as the Example 24(1) using 2-amino-6-methylbenzoic acid and acetyl chloride as the raw materials.

Yield: 53.1%.

¹H-NMR(CDCl₃): δ 2.43(3H, s), 2.79(3H, s), 7.28(1H, d, J=7.9 Hz), 7.37(1H, d, J=7.9 Hz), 7.62(1H, t, J=7.9 Hz).

### (2) 3-Amino-2,5-dimethyl-3,4-dihydroquinazolin-4-one

The title compound was obtained in the same manner as the Example 24(2) under the following reaction condition.

Raw materials: 2,5-dimethyl-4H-3,1-benzoxazin-4-one and hydrazine monohydrate.

Solvent: ethanol.

Reaction: refluxed for 8 hours.

Yield: 45.0%.

¹H-NMR(CDCl₃): δ 2.68(3H, s), 2.87(3H, s), 4.83(2H, s), 7.19(1H, d, J=7.3 Hz), 7.44-7.59(3H, m).

(3) Preparation of 2,5-dimethyl-3-[(5-hydroxy-3-methyl-1-phenylpyrazol-4-yl)methylidene]amino-3,4-dihydroquinazolin-4-one (Compound No. 33)

The title compound was obtained in the same manner as the Example 1(4) using 3-amino-2,5-dimethyl-3,4-dihydroquinazolin-4-one and 3-methyl-5-oxo-1-phenyl-4,5-dihydropyrazole-4-carbaldehyde (compound of Example 1(3)) as the raw materials.

Yield: 64.4%.

¹H-NMR(DMSO-d₆): δ 2.20(3H, s), 2.50(3H, s), 2.77(3H, s), 7.10(1H, t, J=7.3 Hz), 7.27-7.48(4H, m), 7.69(1H, t, J=7.8 Hz), 8.01(2H, d, J=7.9 Hz), 8.11(1H, s).

### Example 34

### Preparation of the Compound of Compound No. 34

### (1) 2,8-Dimethyl-4H-3,1-benzoxazin-4-one

The title compound was obtained in the same manner as the Example 24(1) using 2-amino-3-methylbenzoic acid and acetyl chloride as the raw materials.

Yield: 100%.

¹H-NMR(CDCl₃): δ 2.47(3H, s), 2.54(3H, s), 7.37(1H, t, J=7.8 Hz), 7.61-7.64(1H, m), 8.01-8.04(1H, m).

### (2) 3-Amino-2,8-dimethyl-3,4-dihydroquinazolin-4-one

The title compound was obtained in the same manner as the Example 24(2) under the following reaction condition.

Raw materials: 2,8-dimethyl-4H-3,1-benzoxazin-4-one and hydrazine monohydrate.

Solvent: ethanol.

Reaction: refluxed for 8 hours.

Yield: 37.0%.

¹H-NMR(CDCl₃): δ 2.60(3H, s), 2.71(3H, s), 4.89(2H, brs), 7.32(1H, t, J=7.6 Hz), 7.55-7.65(1H, m), 8.08(1H, dd, J=7.6, 1.0 Hz).

### (3) Preparation of 2,8-dimethyl-3-[(5-hydroxy-3-methyl-1-phenylpyrazol-4-yl)methylidene]amino-3,4-dihydroquinazolin-4-one (Compound No. 34)

The title compound was obtained in the same manner as the Example 1(4) using 3-amino-2,8-dimethyl-3,4-dihydroquinazolin-4-one and 3-methyl-5-oxo-1-phenyl-4,5-dihydropyrazole-4-carbaldehyde (compound of Example 1(3)) as the raw materials.

Yield: 71.8%.

¹H-NMR(DMSO-d₆): δ 2.23(3H, s), 2.50(3H, s), 2.55(3H, s), 7.07(1H, t, J=6.9 Hz), 7.32-7.42(3H, m), 7.67-7.70(1H, m), 7.95-8.10(4H, m).

### Example 35

### Preparation of the Compound of Compound No. 35

### (1) 3-Amino-6-chloro-2-methyl-3,4-dihydroquinazolin-4-one

The title compound was obtained in the same manner as the Example 1(2) using 2-acetamido-5-chlorobenzoic acid methyl ester and hydrazine monohydrate as the raw materials.

Yield: 84.9%.

¹H-NMR(CDCl₃): δ 2.70(3H, s), 4.89(2H, s), 7.58(2H, d, J=8.6 Hz), 7.66(1H, dd, J=8.6, 2.3 Hz), 8.19(1H, d, J=2.3 Hz).

### (2) Preparation of 6-chloro-3-[(5-hydroxy-3-methyl-1-phenylpyrazol-4-yl)methylidene]amino-2-methyl-3,4-dihydroquinazolin-4-one (Compound No. 35)

The title compound was obtained in the same manner as the Example 1(4) using 3-amino-6-chloro-2-methyl-3,4-dihydroquinazolin-4-one and 3-methyl-5-oxo-1-phenyl-4,5-dihydropyrazole-4-carbaldehyde (compound of Example 1(3)) as the raw materials.

Yield: 88.9%.

¹H-NMR(DMSO-d₆): δ 2.18(3H, s), 2.49(3H, s), 7.16(1H, t, J=7.3 Hz), 7.39-7.45(2H, m), 7.72(1H, d, J=8.6 Hz), 7.90-7.98(3H, m), 8.10(1H, d, J=2.3 Hz), 8.19(1H, s).

### Example 36

### Preparation of the Compound of Compound No. 36

### (1) 2-Acetamido-4-chlorobenzoic acid methyl ester

The title compound was obtained in the same manner as the Example 1(1) using 2-amino-4-chlorobenzoic acid methyl ester and acetyl chloride as the raw materials.

Yield: 63.7%.

¹H-NMR(CDCl₃): δ 2.24(3H, s), 3.93(3H, s), 7.05(1H, dd, J=8.6, 2.1 Hz), 7.95(1H, d, J=8.6 Hz), 8.82(1H, d, J=2.1 Hz), 11.10(1H, brs).

### (2) 3-Amino-7-chloro-2-methyl-3,4-dihydroquinazolin-4-one

The title compound was obtained in the same manner as the Example 1(2) using 2-acetamido-4-chlorobenzoic acid methyl ester and hydrazine monohydrate as the raw materials.

Yield: 74.5%.

¹H-NMR(CDCl₃): δ 2.70(3H, s), 4.88(2H, brs), 7.40(1H, dd, J=8.6, 2.0 Hz), 7.63(1H, d, J=2.0 Hz), 8.15(1H, d, J=8.6 Hz).

### (3) Preparation of 7-chloro-3-[(5-hydroxy-3-methyl-1-phenylpyrazol-4-yl)-methylidene]amino-2-methyl-3,4-dihydroquinazolin-4-one (Compound No. 36)

The title compound was obtained in the same manner as the Example 1(4) using 3-amino-7-chloro-2-methyl-3,4-dihydroquinazolin-4-one and 3-methyl-5-oxo-1-phenyl-4,5-dihydropyrazole-4-carbaldehyde (compound of Example 1(3)) as the raw materials.

Yield: 75.5%.

¹H-NMR(DMSO-d₆): δ 2.18(3H, s), 2.49(3H, s), 7.16(1H, t, J=7.4 Hz), 7.42(2H, t, J=7.9 Hz), 7.59-7.63(1H, m), 7.77(1H, d, J=2.3 Hz), 7.97(2H, d, J=7.6 Hz), 8.13-8.19(2H, m).

### Example 37

### Preparation of the Compound of Compound No. 37

### (1) 3-Amino-6-bromo-2-methyl-3,4-dihydroquinazolin-4-one

The title compound was obtained in the same manner as the Example 1(2) using 2-acetamido-5-bromobenzoic acid methyl ester and hydrazine monohydrate as the raw materials.

Yield: 93.7%.

¹H-NMR(CDCl₃): δ 2.70(3H, s), 4.89(2H, s), 7.51(1H, d, J=8.6 Hz), 7.80(1H, dd, J=8.6, 2.2 Hz), 8.36(1H, d, J=2.2 Hz).

### (2) Preparation of 6-bromo-3-[(5-hydroxy-3-methyl-1-phenylpyrazol-4-yl)-methylidene]amino-2-methyl-3,4-dihydroquinazolin-4-one (Compound No. 37)

The title compound was obtained in the same manner as the Example 1(4) using 3-amino-6-bromo-2-methyl-3,4-dihydroquinazolin-4-one and 3-methyl-5-oxo-1-phenyl-4,5-dihydropyrazole-4-carbaldehyde (compound of Example 1(3)) as the raw materials.

Yield: 81.2%.

¹H-NMR(DMSO-d₆): δ 2.18(3H, s), 2.49(3H, s), 7.16(1H, t, J=7.3 Hz), 7.42(2H, d, J=7.9 Hz), 7.65(1H, d, J=8.6 Hz), 7.97(2H, d, J=8.7 Hz), 8.03(1H, dd, J=8.6, 2.3 Hz), 8.18(1H, s), 8.19(1H, d, J=2.3 Hz).

### Example 38

### Preparation of the Compound of Compound No. 38

### (1) 2-Acetamido-4,5-dimethoxybenzoic acid methyl ester

The title compound was obtained in the same manner as the Example 1(1) using 2-amino-4,5-dimethoxybenzoic acid methyl ester and acetyl chloride as the raw materials.

Yield: 55.0%.

¹H-NMR(CDCl₃): δ 2.23(3H, s), 3.89(3H, s), 3.91(3H, s), 3.96(3H, s), 7.45(1H, s), 8.47(1H, s).

### (2) 3-Amino-6,7-dimethoxy-2-methyl-3,4-dihydroquinazolin-4-one

The title compound was obtained in the same manner as the Example 1(2) using 2-acetamido-4,5-dimethoxybenzoic acid methyl ester and hydrazine monohydrate as the raw materials.

Yield: 88.5%.

¹H-NMR(CDCl₃): δ 2.69(3H, s), 3.99(3H, s), 4.00(3H, s), 4.89(2H, s), 7.04(1H, s), 7.52(1H, s).

### (3) Preparation of 6,7-dimethoxy-3-[(5-hydroxy-3-methyl-1-phenylpyrazol-4-yl)-methylidene]amino-2-methyl-3,4-dihydroquinazolin-4-one (Compound No. 38)

The title compound was obtained in the same manner as the Example 1(4) using 3-amino-6,7-dimethoxy-2-methyl-3,4-dihydroquinazolin-4-one and 3-methyl-5-oxo-1-phenyl-4,5-dihydropyrazole-4-carbaldehyde (compound of Example 1(3)) as the raw materials.

Yield: 56.2%.

¹H-NMR(DMSO-d₆): δ 2.22(3H, s), 2.49(3H, s), 3.87(3H, s), 3.90(3H, s), 7.05-7.11(2H, m), 7.31-7.40(3H, m), 8.01-8.07(3H, m).

### Example 39

### Preparation of the Compound of Compound No. 39

### (1) Preparation of 3-amino-2-hydroxymethyl-1,2,3,4-tetrahydroquinazolin-4-one

A powder of 5% palladium on activated carbon (0.08 g) was added to a solution of 3-amino-2-hydroxymethyl-3,4-hihydroquinazolin-4-one (compound of Example 26(2); 0.30 g, 1.57 mmol) in a mixed solvent of tetrahydrofuran/methanol (40 mL+40 mL), and the mixture was stirred for 8 hours under hydrogen atmosphere. After the insoluble matter was filtered off, the residue obtained by evaporation of the solvent under reduced pressure was purified by column chromatography on silica gel(dichloromethane:methanol=99:1→9:1) to give the title compound (0.217 g, 71.5%) as a white solid.

¹H-NMR(DMSO-d₆): δ 3.52-3.60(2H, m), 4.65-4.70(1H, m), 4.90-5.00(3H, m), 6.60-6.65(1H, m), 6.71(1H, d, J=8.3 Hz), 6.86(1H, d, J=1.7 Hz), 7.16-7.23(1H, m), 7.56(1H, dd, J=7.6, 1.3 Hz).

### (3) Preparation of 3-[(5-hydroxy-3-methyl-1-phenylpyrazol-4-yl)methylidene]-amino-2-hydroxymethyl-1,2,3,4-tetrahydroquinazolin-4-one (Compound No. 39)

The title compound was obtained in the same manner as the Example 1(4) using 3-amino-2-hydroxymethyl-1,2,3,4-tetrahydroquinazolin-4-one and 3-methyl-5-oxo-1-phenyl-4,5-dihydropyrazole-4-carbaldehyde (compound of Example 1(3)) as the raw materials.

Yield: 52.3%.

¹H-NMR(DMSO-d₆): δ 2.18(3H, s), 3.64(2H, m), 5.12(1H, m), 5.34(1H, br), 6.68-6.80(2H, m), 7.09-7.15(1H, m), 7.21(1H, s), 7.28-7.42(3H, m), 7.65(1H, d, J=7.9 Hz), 7.96-7.99(2H, m), 8.09(1H, s).

### Example 40

### Preparation of the Compound of Compound No. 40

3-[(5-Hydroxy-3-methyl-1-phenylpyrazol-4-yl)methylidene]amino-2-hydroxym ethyl-1,2,3,4-tetrahydroquinazolin-4-one (Compound No. 39) was dissolved in 50% aqueous ethanol, and 10 mg/mL solution was prepared from the solution. Optical resolution of 2 mL of the solution was carried out by high performance liquid chromatography using chiral column.

<Condition>

Pump: SHIMADZU LC-7A.

Detector: SHIMADZU SPD-7A.

Wavelength for detection: 254 nm.

Column: SHISEIDO chiral CD-Ph 4.6 mm×250 nm; particle size 5 μm.

Mobile Phase: 60% aqueous methanol

Flow rate: 0.6 mL/min.

Injection volume per time: 100 μL.

An optically active form of 3-[(5-hydroxy-3-methyl-1-phenylpyrazol-4-yl)-methylidene]amino-2-hydroxymethyl-1,2,3,4-tetrahydroquinazolin-4-one (8.1 mg, 99.9% ee.) was obtained from the first fraction.

### Example 41

### Preparation of the Compound of Compound No. 41

An optically active form of 3-[(5-hydroxy-3-methyl-1-phenylpyrazol-4-yl)-methylidene]amino-2-hydroxymethyl-1,2,3,4-tetrahydroquinazolin-4-one (8.2 mg, 94.4% ee.) was obtained from the second fraction in the Example 40.

This compound is an enantiomer of the compound of Compound No. 40.

### Example 42

### Preparation of the Compound of Compound No. 42

The title compound was obtained in the same manner as the Example 1(4) under the following reaction condition.

Raw materials: 3-amino-2-methyl-3,4-dihydroquinazolin-4-one (compound of Example 1(2)) and diphenylacetaldehyde.

Solvent: ethanol.

Reaction: refluxed for 12 hours.

Yield: 34.2%.

¹H-NMR(CDCl₃): δ 2.50(3H, s), 5.27(1H, d, J=7.1 Hz), 7.25-7.75(13H, m), 8.23(1H, dt, J=8.0, 0.8 Hz), 8.75(1H, d, J=7.1 Hz).

### Example 43

### Preparation of the Compound of Compound No. 43

The title compound was obtained in the same manner as the Example 1(4) under the following reaction condition.

Raw materials: 3-amino-2-methyl-3,4-dihydroquinazolin-4-one (compound of Example 1(2)) and β-phenylcinnamaldehyde.

Solvent: ethanol.

Reaction: refluxed for 20 hours.

Yield: 15.7%.

¹H-NMR(CDCl₃): δ 2.60(3H, s), 7.06 (1H, d, J=10.0 Hz), 7.33-7.48(11H, m), 7.62(1H, d, J=8.0 Hz), 7.68-7.73(1H, m), 8.24(1H, dd, J=8.0, 1.6 Hz), 8.43(1H, d, J=10.0 Hz).

### Example 44

### Preparation of the Compound of Compound No. 44

### (1) Preparation of 3-(1-adamantyl)-1-phenyl-4,5-dihydropyrazol-5-one

The title compound was obtained in the same manner as the Example 6(1) using ethyl 3-(1-adamantyl)-3-oxopropionate and phenylhydrazine as the raw materials.

Yield: 80.7%.

¹H-NMR(CDCl₃): δ 1.72-2.07(15H, m), 3.42(2H, s), 7.14-7.19(1H, m), 7.35-7.42(2H, m), 7.87-7.92 (2H, m).

### (2) Preparation of 3-(1-adamantyl)-5-oxo-1-phenyl-4,5-dihydropyrazole-4-carbaldehyde

The title compound was obtained in the same manner as the Example 1(3) using 3-(1-adamantyl)-1-phenyl-4,5-dihydropyrazol-5-one as the raw material.

Yield: 47.1%.

¹H-NMR(CDCl₃): δ 1.78-2.10(15H, m), 7.27-7.32(1H, m), 7.43-7.81(2H, m), 7.84-7.87(2H, m), 9.83(1H, s).

### (3) Preparation of 3-(1-adamantyl)-5-hydroxy-1-phenylpyrazol-4-yl}-methylidene)amino-2-methyl-3,4-dihydroquinazolin-4-one (Compound No. 44)

The title compound was obtained in the same manner as the Example 1(4) under the following reaction condition.

Raw materials: 3-amino-2-methyl-3,4-dihydroquinazolin-4-one (compound of Example 1(2)) and 3-(1-adamantyl)-5-oxo-1-phenyl-4,5-dihydropyrazole-4-carbaldehyde.

Solvent: ethanol.

Reaction: stirred at room temperature overnight.

Yield: 32.3%.

¹H-NMR(CDCl₃):δ 1.77-1.82(6H, m), 2.05-2.08(9H, m), 2.66(3H, s), 7.18(1H, t, J=7.4 Hz), 7.39(1H, d, J=8.0 Hz), 7.42(1H, d, J=7.4 Hz), 7.51(1H, t, J=8.0 Hz), 7.68(1H, d, J=8.0 Hz), 7.78-7.83(1H, m), 7.96(1H, s), 7.97(2H, d, J=8.0 Hz), 8.27(1H, d, J=8.5 Hz).

### Example 45

### Preparation of the Compound of Compound No. 45

### (1) Preparation of 1-(4-tert-butylphenyl)-3,3-bis(morpholino)prop-2-en-1-one

4-tert-Butylbenzoyl chloride (1.95 mL, 10.0 mmol) was added dropwise for 1 hour to a solution of 1,1-bis(N-morpholino)ethylene (1.98 g, 10.0 mmol and triethylamine (1.56 mL, 11.2 mmol) in chloroform under argon atmosphere at 0° C., and the mixture was stirred at room temperature overnight. 2N sodium hydroxide was added to the reaction mixture and the mixture was extracted with dichloromethane. The dichloromethane layer was washed with brine and dried over anhydrous sodium sulfate. The residue obtained by evaporation of the solvent under reduced pressure was washed with diethyl ether to give the title compound (1.74 g, 48.7%) as a white crystal.

¹H-NMR(CDCl₃): δ 1.33(9H, s), 3.28-3.36 (8H, m), 3.72-3.82 (8H, m), 5.13(1H, s), 7.42(2H, d, J=8.4 Hz), 7.79(2H, d, J=8.4 Hz).

### (2) Preparation of ethyl 4-tert-butylbenzoylacetate

Trifluoroacetic acid (4 drops) was added to a solution of 1-(4-tert-butylphenyl)-3,3-bis(morpholino)prop-2-en-1-one (1.08 g, 3.0 mmol) in ethanol (50.0 mL), and the mixture was refluxed for 49 hours. After the reaction mixture was cooled to room temperature, the residue obtained by evaporation of the solvent under reduced pressure was purified by column chromatography on silica gel(n-hexane:dichloromethane=1:2) to give the title compound (327 mg, 43.9%) as a colorless oil.

¹H-NMR(CDCl₃): δ 1.26(3H, t, J=7.1 Hz), 1.34(9H, s), 3.97(2H, s), 4.22(2H, q, J=7.1 Hz), 7.49(2H, d, J=8.2 Hz), 7.89(2H, d, J=8.2 Hz).

### (3) Preparation of 3-(4-tert-butylphenyl)-1-phenyl-4,5-dihydropyrazol-5-one

The title compound was obtained in the same manner as the Example 6(1) using ethyl 4-tert-butylbenzoylacetate and phenylhydrazine as the raw materials.

Yield: 84.2%.

¹H-NMR(CDCl₃): δ 1.35(9H, s), 3.85(2H, s), 7.22(2H, t, J=7.4 Hz), 7.41-7.49(3H, m), 7.71(2H, d, J=8.2 Hz), 7.99(2H, d, J=8.8 Hz).

### (4) Preparation of 3-(4-tert-butylphenyl)-5-oxo-1-phenyl-4,5-dihydropyrazol-4-carbaldehyde

The title compound was obtained in the same manner as the Example 1(3) using 3-(4-tert-butylphenyl)-1-phenyl-4,5-dihydropyrazol-5-one as the raw material.

Yield: 64.6%.

¹H-NMR(DMSO-d₆): δ 1.32(9H, s), 7.25-7.30(1H, m), 7.45-7.50(4H, m), 7.82(2H, d, J=8.2 Hz), 7.90(2H, d, J=7.4 Hz), 9.54(1H, s).

### (5) Preparation of 3-(4-tert-butylphenyl)-5-hydroxy-1-phenylpyrazol-4-yl}-methylidene)amino-2-methyl-3,4-dihydroquinazolin-4-one (Compound No. 45)

The title compound was obtained in the same manner as the Example 1(4) under the following reaction condition.

Raw materials: 3-amino-2-methyl-3,4-dihydroquinazolin-4-one (compound of Example 1(2)) and 3-(4-tert-butylphenyl)-5-oxo-1-phenyl-4,5-dihydropyrazol-4-carbaldehyde.

Solvent: ethanol.

Reaction: stirred at room temperature overnight.

Yield: 45.9%.

¹H-NMR(DMSO-d₆): δ 1.29(9H, s), 2.54(3H, s), 7.21(1H, t, J=7.4 Hz), 7.45-7.50(5H, m), 7.65-7.68(3H, m), 7.84-7.89(1H, m), 8.07-8.14(3H, m), 8.40(1H, s).

### Example 46

### Preparation of the Compound of Compound No. 46

The title compound was obtained in the same manner as the Example 1(4) under the following reaction condition.

Raw materials: 3-amino-2-methyl-3,4-dihydroquinazolin-4-one (compound of Example 1(2)) and 4-(diphenylamino)benzaldehyde.

Solvent: ethanol.

Reaction: refluxed for 43 hours.

Yield: 6.0%.

¹H-NMR(CDCl₃): δ 2.49(3H, s), 6.98(2H, d, J=8.8 Hz), 7.16-7.19(6H, m), 7.38-7.43(4H, m), 7.49-7.54(1H, m), 7.65(1H, d, J=7.7 Hz), 7.79-7.82(3H, m), 8.14(1H, dd, J=7.8, 1.3 Hz), 8.75(1H, s).

### Example 47

### Preparation of the Compound of Compound No. 47

The title compound was obtained in the same manner as the Example 1(4) under the following reaction condition.

Raw materials: 3-amino-2-methyl-3,4-dihydroquinazolin-4-one (compound of Example 1(2)) and 9-ethylcarbazole-3-carbaldehyde.

Solvent: ethanol.

Reaction: refluxed for 4 hours.

Yield: 94.5%.

¹H-NMR(CDCl₃): δ 1.43(3H, t, J=7.1 Hz), 2.81(1H, t, J=5.9 Hz), 3.90-3.98(1H, m), 4.06-4.13(1H, m), 4.35(2H, q, J=7.1 Hz), 4.94(1H, d, J=2.7 Hz), 5.26-5.31(1H, m), 6.74(1H, dd, J=8.2, 0.5 Hz), 6.88-6.93(1H, m), 7.23-7.49(5H, m), 7.86(1H, dd, J=8.6, 1.6 Hz), 7.97(1H, dd, J=8.0, 1.6 Hz), 8.03-8.12(1H, m), 8.41(1H, d, J=1.6 Hz), 9.31(1H, s).

### TEST EXAMPLE

### Test Example 1

### Measurement of Inhibitory Activity Against Spleen-Type Prostaglandin D2 Synthase

A buffer solution of 100 mM Tris (hydroxymethyl)aminomethane/HCl buffer (pH8.0) containing glutathione (0.1 mM) and human hematopoietic prostaglandin D2 synthase (adequate quantity) was preincubated at 25° C. for 5 minutes in the presence or absence of a test compound, and then 14C labeled prostaglandin H2 ([14C]PGH2) (10 μM) was added, and the buffer solution was further incubated at 25° C. for one minute. A mixture of ether, methanol, and citric acid was added to the reaction mixture, and the ether layer was developed on silica gel thin-layer chromatography (TLC) (eluent=ether: methanol: acetic acid=90:2:1), and the PGD2 produced was measured by exposure to an imaging plate. The enzyme inhibitory rates of test compounds were calculated assuming the amount of production of PGD2 without the test compound being 100%. The results are shown below.

| Compound | Inhibitory Ratio of Prostaglandin D2 (PGD2) Synthase (%) | |
|---|---|---|
| Number | Drug Concentration 30 μM | Drug Concentration 10 μM |
| 1 | 76 | 50 |
| 3 | N.T. | 13 |
| 5 | 70 | 43 |
| 6 | N.T. | 18 |
| 7 | 105 | 96 |
| 10 | 48 | 24 |
| 11 | N.T. | 45 |
| 12 | N.T. | 46 |
| 13 | 54 | 3 |
| 14 | 27 | −2 |
| 15 | 53 | 20 |
| 16 | 81 | 50 |
| 17 | N.T. | 29 |
| 18 | 37 | 23 |
| 19 | 50 | 6 |
| 20 | 83 | 37 |
| 21 | 51 | 13 |
| 22 | 44 | 14 |
| 26 | 71 | 86 |
| 27 | 13 | −4 |
| 28 | 30 | 19 |
| 29 | 40 | 20 |
| 30 | 71 | 51 |
| 31 | 26 | 23 |
| 32 | 15 | 12 |
| 33 | 61 | 27 |
| 34 | 84 | 71 |
| 35 | 68 | 15 |
| 36 | 60 | 39 |
| 37 | 56 | 15 |
| 38 | 51 | 20 |
| 39 | 46 | 77 |

N.T.: Not Tested

### Test Example 2

### Induction of Hematopoietic Prostaglandin D Synthase and DP Receptor in Hereditary Demyelinating Disease

Using a model mouse of human Krabbe disease Twitcher which is a Galactosylceramidase deficiency (Brain Research, (Netherlands), 1980, Vol. 202, No. 2, p. 479-483; Brain; A Journal of Neurology, (England), 1980, Vol. 103, No. 3, p. 695-710; Journal of Neurochemistry, (England), 1996, Vol. 66, No. 3, p. 1118-1124; Journal of Neuropathology and Experimental Neurology, (USA), 1999, Vol. 58, No. 6, p. 644-653), changes of mRNA of H-PGDS and DP receptor accompanied by brain damage by hereditary demyelination were quantitatively measured by a quantitative RT-PC method. As a result, the expression amounts of mRNA of H-PGDS and DP receptor were increased together with the brain damage by hereditary demyelination. By an immunohistostaining method, it was identified that H-PGDS expresses in microglial cells, Ameboid cells and macrophage cells which accumulate in the tissue region where demyelination is advanced, and DP receptor expresses in the activated astroglial cells that distribute in the vicinity of tissues where demyelination is advanced.

### Test Example 3

### Induction of Hematopoietic Prostaglandin D Synthase and DP Receptor in Autoimmune Demyelinating Disease

In the experimental autoimmune encephalomyelitis mouse which is a model of human multiple sclerosis (Cellular Immunology, Vol. 191, 97-104, 1999; and Nature Reviews; Neuroscience, Vol. 3, 291-301, 2002), the expression amounts of mRNA of H-PGDS and DP receptor were measured by the quantitative RT-PCR method. As a result, the expression amounts of mRNA of H-PGDS and DP receptor showed increase in relation to the brain damage by demyelination. In the observation of the immunohistostaining method, H-PGDS expressed in microglial cells, Ameboid cells and macrophage cells which accumulate in the tissue region where demyelination is advanced.

### Test Example 4

### Induction of Hematopoietic Prostaglandin D Synthase and DP Receptor in Traumatic Stimulation

Using traumatic brain cortex disorder (Stab wound) model (Brain Research, Vol. 883, 87-97, 2000; Journal of Neurochemisry, Vol. 73, 812-820, 1999), expression of mRNA of H-PGDS and DP receptor in brain damage was examined. As a result, H-PGDS reached the maximum value two days after the injury, and DP receptor continuously increased from day 2 to day 8. Induction of H-PGDS occurred 24 hours after the injury in the microglia cells and maclophage cells which accumulate around the injured region, expression of GFAP and DP receptor increased in the astroglia cells around the injured region, and these phenomena sustained 8 days later.

### Test Example 5

### Aggravation of Traumatic Brain Damage by Mass Expression of Human Hematopoietic Prostaglandin D Synthase

In the Stab wound model using human H-PGDS mass expression transgenic mouse (see, the pamphlets of International Publication WO 01/24607), accumulations of macrophage in the injured region and activation of astroglia cells, examined immunohistochemically using anti GFAP antibody, are remarkable compared with the wild type mouse, and the recovery was delayed.

### Test Example 6

### Inhibition of Activation of Astroglia Cell in Hereditary Demyelinating Disease by Administration of Hematopoietic Prostaglandin D Synthase Inhibitor

HQL-79 as an H-PGDS inhibitor was administered subcutaneously to the Twitcher mouse at a dose of 30 mg/kg/day for 14 days. As a result, activation of astroglia cells was inhibited, and at the same time, the expression of DP receptor in the astroglia cells was decreased.

### Test Example 7

### Inhibition of DP Receptor and Recovery Promotion in Traumatic Brain Injury by Administration of Hematopoietic Prostaglandin D Synthase Inhibitor

HQL-79 as an H-PGDS inhibitor was administered orally to the Stab wound mouse with a dose of 30 mg/kg/day for 4 days. As a result, DP receptor mRNA in the tissue damaged region decreased, and a recovery promotion of the brain damage was recognized.

### Test Example 8

### Prostaglandin D2 Production Inhibition Test Using Cells

RBL-2H3, a rat basophilic leukemia cell to express hematopoietic prostaglandin D synthase, was inoculated in a 24 well plate and incubated overnight. After replacement with a medium in the presence or absence of a test compound, preincubation was carried out at 37° C. for one hour. After washing of the cells with PBS(-), the medium was replaced with a Hepes buffer solution (pH7.4) in the presence or absence of the test compound, and incubation was continued at 37° C. for 15 minutes. Then, calcium ionophore (A23187) at a final concentration of 2.5 μM was added, and incubation was carried out at 37° C. for 15 minutes to induce the production of PGD2. The supernatant of the cells was collected, and the amount of PGD2 flowed out in the supernatant was measured by EIA kit by Cayman. The inhibitory ratio of the production of PGD2 by the test compound was calculated assuming the amount of PGD2 produced by A23187 in the absence of the test compound as being 100%. The results are shown below.

| | Inhibitory Ratio of Prostaglandin |
|---|---|
| Compound | D2 (PGD2) Production (%) |
| Number | Drug Concentration 10 μM |
| 1 | 26 |
| 3 | 21 |
| 7 | 50 |
| 17 | 21 |
| 20 | 15 |
| 24 | 59 |
| 25 | 47 |
| 30 | 28 |
| 37 | 32 |
| 39 | 70 |
| 40 | 74 |
| 41 | 80 |
| 42 | 89 |
| 43 | 82 |
| 44 | 100 |
| 45 | 100 |
| 46 | 71 |
| 47 | 77 |

### INDUSTRIAL APPLICABILITY

The medicaments of the present invention have a strong inhibitory activity against hematopoietic PGD2 synthase. Therefore, the medicaments of the present invention are useful for prevention and/or therapeutic treatment of diseases such as allergy, allergic inflammation and asthma. Further, the medicaments of the present invention are also useful as those having actions such as a prevention of aggravation and/or an improvement of prognosis of brain damage, a protection against tissue damage, a regulation of estrous cycle, a regulation of sleep, a thermoregulation, an analgesia, and a regulation of olfaction.

1. A medicament having inhibitory activity against hematopoietic prostaglandin D2 (PGD2) synthase, which comprises as an active ingredient a substance selected from the group consisting of a compound represented by the following general formula (I) and a pharmacologically acceptable salt thereof, and a hydrate thereof and a solvate thereof: 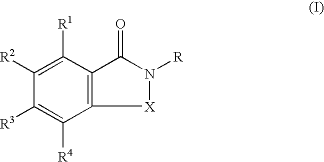
wherein X represents a group represented by the formula —N═C(R⁵)— (wherein a bond at the left end binds to the benzene ring and a bond at the right end binds to the nitrogen atom), or the formula —NH—CH(R⁵)— (wherein a bond at the left end binds to the benzene ring and a bond at the right end binds to the nitrogen atom), R¹, R², R³, and R⁴ independently represent a hydrogen atom, a halogen atom, a C₁ to C₆ alkyl group which may be substituted, or a hydroxy group which may be substituted,
R⁵ represents a C₁ to C₆ alkyl group which may be substituted, or a C₆ to C₁₀ aryl group which may be substituted,
R represents an amino group which may be substituted.

2. The medicament according to claim 1, wherein R is a group represented by the following general formula (II): 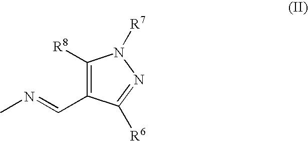
wherein R⁶ represents a C₁ to C₁₀alkyl group which may be substituted, or a C₆ to C₁₀ aryl group which may be substituted,
R⁷ represents a C₁ to C₆ alkyl group which may be substituted, or a C₆ to C₁₀ aryl group which may be substituted,
R⁸ represents a halogen atom, hydroxy group, or a C₁ to C₆ alkoxy group which may be substituted.

3. The medicament according to claim 1, wherein X is a group represented by the formula —N═C(R⁵)— (wherein a bond at the left end binds to the benzene ring and a bond at the right end binds to the nitrogen atom).

4. The medicament according to claim 1, wherein R¹, R², R³, and R⁴ independently represent a hydrogen atom, a halogen atom, a C₁ to C₆ alkyl group, or a C₁ to C₆ alkoxy group.

5. The medicament according to claim 1, wherein R⁵ is a C₁ to C₆ alkyl group which may be substituted with a group selected from the following substituent group α-1, or a phenyl group which may be substituted with a group selected from the following substituent group α-1.
[Substituent Group α-1] hydroxy group, C₁ to C₆ alkoxy group

6. The medicament according to claim 2, wherein R⁶ is a C₁ to C₁₀ alkyl group which may be substituted with a group selected from the following substituent group α-2, or a phenyl group which may be substituted with a C₁ to C₆ alkyl group.
[Substituent Group α-2] halogen atoms, carboxy group, carbamoyl group, C₁ to C₆ alkoxycarbonyl group

7. The medicament according to claim 2, wherein R⁷ is a C₁ to C₆ alkyl group, or a phenyl group which may be substituted with a group selected from the following substituent group α-3.
[Substituent Group α-3] halogen atoms, C₁ to C₆ alkyl group, C, to C₆ alkoxy group, nitro group

8. The medicament according to claim 2, wherein R⁸ is a halogen atom, hydroxy group, or a C₁ to C₆ alkoxyl group which may be substituted with a group selected from the following substituent group α-4.
[Substituent Group α4] carboxy group, C₁ to C₆ alkoxycarbonyl group.

9. The medicament according to claim 1, having one or more actions selected from the group consisting of antiallergic action, antiallergic inflammation, and antiasthmatic action.

10. The medicament according to claim 1, having an action of preventing the aggravation of brain damage, and/or an action of improving the prognosis of brain damage.

11. The medicament according to claim 1, having one or more actions selected from the group consisting of an action of regulating estrous cycle, an action of regulating sleep, an action of thermoregulation, an analgesic action, and an action of regulating olfaction.

12. A compound represented by the general formula (I-1) or a salt thereof, or a hydrate thereof or a solvate thereof: 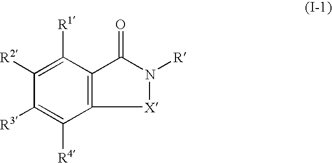
wherein X′ represents a group represented by the formula —N═C(R⁵′)— (wherein a bond at the left end binds to the benzene ring and a bond at the right end binds to the nitrogen atom), or the formula —NH—CH(R⁵′)— (wherein a bond at the left end binds to the benzene ring and a bond at the right end binds to the nitrogen atom),
R¹′, R²′, R³′, and R⁴′ independently represent a hydrogen atom, a halogen atom, a C₁ to C₆ alkyl group which may be substituted, or a hydroxy group which may be substituted,
R⁵′ represents a C₁ to C₆ alkyl group which may be substituted, or a C₆ to C₁₀ aryl group which may be substituted,
R′ represents an amino group which may be substituted,
provided that the compounds represented by the following compound group β are excluded.

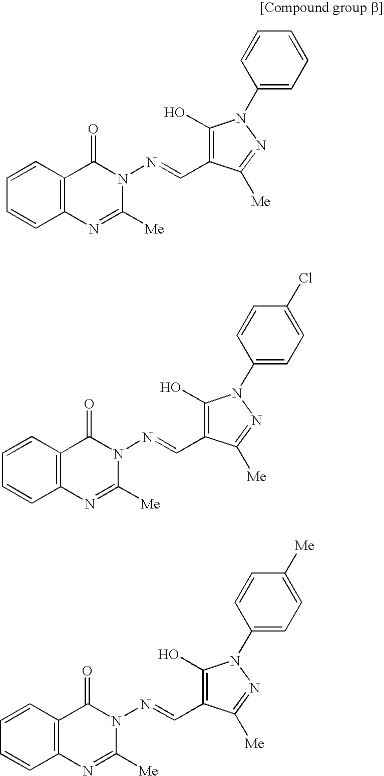

13. The compound according to claim 12 or a salt thereof, or a hydrate thereof or a solvate thereof, wherein R′ is represented by the following general formula (II-1): 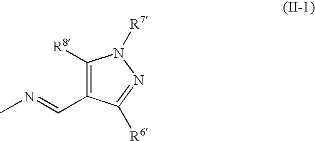
wherein R⁶′ represents a C₁ to C₁₀ alkyl group which may be substituted, or a phenyl group which may be substituted with a C₁ to C₆ alkyl group,
R⁷′ represents a C₁ to C₆ alkyl group which may be substituted, or a C₆ to C₁₀ aryl group which may be substituted,
R⁸′ represents a halogen atom, hydroxy group, or a C₁ to C₆ alkoxy group which may be substituted.

14. The medicament according to claim 2, wherein X is a group represented by the formula —N═C(R⁵)— (wherein a bond at the left end binds to the benzene ring and a bond at the right end binds to the nitrogen atom).

15. The medicament according to claim 14, wherein R¹, R², R³, and R⁴ independently represent a hydrogen atom, a halogen atom, a C₁ to C₆ alkyl group, or a C₁ to C₆ alkoxy group.

16. The medicament according to claim 15, wherein R⁵ is a C₁ to C₆ alkyl group which may be substituted with a group selected from the following substituent group α-1, or a phenyl group which may be substituted with a group selected from the following substituent group α-1.
[Substituent Group α-1] hydroxy group, C₁ to C₆ alkoxy group

17. The medicament according to claim 16, wherein R⁶ is a C₁ to C₁₀alkyl group which may be substituted with a group selected from the following substituent group α-2, or a phenyl group which may be substituted with a C₁ to C₆ alkyl group.
[Substituent Group α-2] halogen atoms, carboxy group, carbamoyl group, C₁ to C₆ alkoxycarbonyl group

18. The medicament according to claim 17, wherein R⁷ is a C₁ to C₆ alkyl group, or a phenyl group which may be substituted with a group selected from the following substituent group α-3.
[Substituent Group α-3] halogen atoms, C₁ to C₆ alkyl group, C₁ to C₆ alkoxy group, nitro group

19. The medicament according to claim 18, wherein R⁸ is a halogen atom, hydroxy group, or a C₁ to C₆ alkoxyl group which may be substituted with a group selected from the following substituent group α-4.
[Substituent Group α-4] carboxy group, C₁ to C₆ alkoxycarbonyl group.

20. The medicament according to claim 2, wherein R¹, R², R³, and R⁴ independently represent a hydrogen atom, a halogen atom, a C₁ to C₆ alkyl group, or a C₁ to C₆ alkoxy group.
